(19) United States
(12) Patent Application Publication
(10) Pub. No.: US 2010/0075993 A1
(43) Pub. Date: Mar. 25, 2010

(54) SMALL MOLECULE INHIBITORS AGAINST WEST NILE VIRUS REPLICATION

(75) Inventors: Baohua Gu, Malvern, PA (US); Tim Block, Doylestown, PA (US); Andy Cuconati, Oreland, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: DREXEL UNIVERSITY, Philadelphia, PA (US)

(21) Appl. No.: 11/993,592

(22) PCT Filed: Jun. 30, 2006
(86) PCT No.: PCT/US06/25433

§ 371 Date: Dec. 21, 2007

Related U.S. Application Data
(60) Provisional application No. 60/695,770, filed on Jun. 30, 2005.

(51) Int. Cl. A61K 31/519; C07D 235/02; C07D 401/12; C07D 487/04; A61K 31/4162; A61K 31/47; A61K 31/517; A61P 31/12

(52) U.S. Cl. 514/259.1; 548/304.1; 546/211; 544/284; 544/281; 514/405; 514/313; 514/266.2

(57) ABSTRACT

The invention provides flavivirus replication inhibitors of WNV and other flaviviruses, such as, for example, JEV, SLEV, AV, KV, JV, CV, YV, TBEV, DENV-1, DENV-2, DENV-3, DENV-4, YFV and MVEV. The invention further provides pharmaceutical compositions including one or more such flavivirus replication inhibitors. The invention further provides methods of treatment, prevention, inhibition or amelioration of one or more diseases associated with flavivirus replication including administering one or more such inhibitors or pharmaceutical compositions.

### BACKGROUND OF THE INVENTION

1. Field of Invention

This invention relates to the field of antiviral compounds and compositions for the treatment against flaviviral infections. More specifically, it relates to novel flavivirus replication inhibitors and pharmaceutical compositions and the use thereof to treat disorders caused by West Nile virus such as viral encephalitis, and the like, and other emerging flaviviruses, such as, for example, JEV, SLEV, AV, KV, JV, CV, YV, TBEV, DENV-1, DENV-2, DENV-3, DENV-4, YFV and MVEV.

2. Description of Related Art

West Nile virus (WNV) is a mosquito-borne virus that has been introduced to the U.S. in 1999 (1, 2, 10). Since the initial outbreak, there have been cases reported in all but two states in the U.S. WNV has increasingly become a public health threat, causing hundreds of deaths and tens of thousands of infections (7). Although there has been progress in vaccine development to prevent WNV encephalitis in humans (12), there is still no effective vaccine or antiviral drug therapy (7). Currently, the only available treatment is supportive, and the only existing means of prevention is mosquito control, which is also of limited success. It is also considered to be an agent of bioterrorism concern (6), and therefore, safe and effective antiviral drugs to treat WNV infection are urgently needed.

WNV is a positive, single stranded RNA virus (4). It belongs to the Flaviviridae family, and Flavivirus genus. Many flaviviruses are significant human pathogens. In addition to WNV, this flavivirus sero-complex includes Japanese encephalitis virus (JEV), St. Louis encephalitis (SLEV), Alfuy virus (AV), Koutango virus (KV), Kunjin virus (JV), Cacipacore virus (CV), Yaounde virus (YV), and Murray Valley encephalitis virus (MVEV). The Flaviviridae family also includes the Tick-borne encephalitis virus (TBEV), Dengue virus (including the four serotypes of: DENV-1, DENV-2, DENV-3, and DENV-4), and the family prototype, Yellow Fever virus (YFV).

Flaviviruses are the most significant group of arthropod-transmitted viruses in terms of global morbidity and mortality. A combined toll of hundreds of millions of infections around the world annually coupled with the lack of sustained mosquito control measures, has distributed flaviviruses throughout the tropics, subtropics, and temperate areas. As a result, over half the world's population is at risk for flaviviral infection. Further, modern jet travel and human migration have raised the potential for global spread of these pathogens.

Strains of WNV are categorized into two different phylogenetic lineages, namely, lineage I and II, which share 75% nucleotide sequence identity (Lanciotti, R et al, (2002) Virology 298:96-105). Lineage I strains have been isolated from human and equine epidemic outbreaks from around the world and constitute the main form of human pathogen. Sequence analysis indicates that the current epidemic strain in North America belongs to lineage I. Lineage II strains are rarely isolated from humans and are geographically restricted primarily to sub-Saharan Africa and Madagascar. The differences in disease patterns of lineage I and II strains are postulated to be the result of differences in vector competence (host compatibility), virulence, and transmission cycles of the strains, as well as, host immunity (Beasley, D. W. C. et al, (2001) International Conference on the West Nile Virus, New York Academy of Science Poster Section 1:5). Sequence analysis showed that the strain in North America is closely related to other human epidemic strains isolated from Israel, Romania, Russia, and France, all of which belong to lineage I (Lanciotti, R. et al. (1999) Science 286:2333-2337).

The flavivirus genome, including the genome of WNV, is a single positive-sense RNA of approximately 10,500 nucleotides containing short 5′ and 3′ untranslated regions (UTR), a single long open reading frame (ORF), a 5′ cap region, and a non-polyadenylated 3′ terminus. The entire genome is transcribed as a single polycistronic messenger RNA molecule, which is then translated as a polyprotein. Individual proteins are subsequently produced by proteolytic processing of the polyprotein, which is directed by viral and host cell proteases (Chambers, T. J. et al, (1990) Ann. Rev. Microbiol. 44: 649-688; Lindenbach, B. D. and C. M. Rice, (2001) In D. M. Knipe and P. M. Howley (ed), Fields virology, 4.sup.th ed., vol. 1. Lippincott Williams & Wilkins, Philadelphia, Pa.).

During the replication cycle of flaviviruses, especially WNV, synthesis of positive and negative (hereafter referred to as plus (+) and minus (−), respectively) sense RNAs is asymmetric. In the case of WNV, plus-sense RNAs are produced in 10- to 100-fold excess over minus-sense RNA. Regulatory sequences in the 3′ UTR are believed to function as a promoter for initiation of minus-strand RNA synthesis. Deletion of this region ablates viral infectivity (Brinton, M. A. et al, (1986) Virology 162: 290-299; Proutski, V., et al (1997) Nucleic Acids Res. 25: 1194-1202; Rauscher, S., et al (1997) RNA 3: 779-791).

WVN genome is 12 kilobases in length and has a 5′ and 3′ non-translated region (NTR). The coding sequences specify a single polyprotein, which is proteolytically processed into approximately a dozen functional proteins by both viral and cellular proteases (5). The genes for structural Proteins, namely capsid (C), membrane (M; which exists in cells as its precursor, prM), and envelope (E) are located in the 5′ region of the genome, where those for the nonstructural proteins (NS), namely, NS1, NS2A, NS2B, NS3, NS4A, NS4B, and NS5 (5) are located at the 3′ portion of the genome. WNV can infect many cell types and produce cytopathic plaques. However, due to the highly infectious nature, infections must be carried out in BL3 labs, limiting the use of the plaque assay to screen large number of compounds.

The development of therapeutic drugs and/or vaccines to treat and/or immunize against WNV and other flavivirus infections is urgently needed and of great importance to global public health. To achieve this goal, high-throughput screening assays were developed to facilitate the identification of novel chemotherapeutics effective against flaviviruses or vaccines capable of establishing a protective immune response to flaviviruses (see U.S. Patent Application Publication No. 2005/0058987 to Shi et al.). Two general strategies to be adapted for the screening and identification of novel chemotherapeutic antiflaviviral compounds and/or vaccines are based on biochemical and genetic approaches.

Assays for screening antiviral compounds that are based on biochemical approaches typically involve testing compounds for activities that limit or inhibit viral enzymes or proteins that are essential for viral propagation. For example, NS3, which has protease, helicase and NTPase activity, and NS5, which has an RNA-dependent RNA polymerase and methyltransferase activity, are key components of viral replication complex and thus, are ideal targets for antiviral screening. Further, three-dimensional structures of viral proteins, if available, can afford the possibility for rational design of drugs that will inhibit their activity, i.e., designing drugs based on the knowledge of the structure and shape of the active sites of the protein. For example, the crystal structures of the DENV NS3 protease domain and NS5 cap methyltransferase fragment have been solved and thus, the possibility of rationally designing small molecules to inhibit the active sites of NS3 and NS5 is feasible. Although biochemical approaches are capable of identifying potential viral inhibitors, they are limited in their overall efficiency since only a single enzyme or protein can be tested for any potential assay. Thus, individual assays would be required to screen for inhibitors of each given viral target protein.

In contrast, assays utilizing a genetic approach, which are usually cell-based, offer a number of advantages over biochemical approaches. One major advantage of a genetic approach based assay is that multiple viral protein targets can be analyzed simultaneously. A second major advantage is that, since genetic assays involve the use of living cells and the uptake of compounds therein, the screening assay is administered in a more authentic therapeutic environment. Accordingly, inhibitors identified through cell-based assays typically have a higher success rate in subsequent animal experiments.

A cell-based assay available for screening for flaviviral inhibitors involves the infection of cultured cells with virus and the subsequent monitoring for potential inhibition in the presence of a potential inhibitor through observation or quantification of cytopathic effects (J. D. Money et al., Antiviral Res (2002) 55:107-116; 1. Jordan, J. Infect. Dis. (2000) 182:1214-1217) or quantification of viral RNA by reverse transcriptase (RT)-PCR (S. F. Wu, J. Virol. (2002) 76:3596-3604). These assays are highly labor-intensive and impossible to use when screening compound libraries in large quantities.

Genetic high-throughput cell-based screening assays for the rapid screening and identification of potential inhibitors from compound libraries utilizing cDNA clones of RNA viruses are preferred screening tools for identifying potential inhibitors.

For example, two kinds of reverse genetics systems, full-length infectious cDNA clones and replicons, have been developed for a number of flaviviruses (A. A. Khromykh, et al., J. Virol. (1997) 71:1497-1505; M. S. Campbell, et at, Virol. (2000) 269:225-237; R. J. Hurrelbrink, et al., J. Gen. Virol. (1999) 80:3115-3125; M. Kapoor, et at, Gene (1995) 162:175-180; A. A. Khromykh et al., J. Virol. (1994) 68:4580-4588; C. J. Lai et al., Proc. Natl. Acad. Sci. U.S.A. (1991) 88:5139-5143; C. W. Mandl et al., J. Gen. Virol. (1997) 78:1049-1057; C. M. Rice et al., Science (1985) 229:726-733; H. Sumiyoshi et al., J. Virol. (1992) 66:5425-5431; S. Polo et. al., J. Virol. (1997) 71:5366-5374), including lineage II WNV (V. F. Yamshchikov et al., Virology (2001) 281:294-304). Reporter genes can be engineered into the reverse genetics systems to allow for the monitoring of viral replication levels in the presence of potential inhibitors.

U.S. Patent Application Publication No. 2005/0058987 to Shi et al. describes high-throughput cell-based assays for the rapid screening and identification of potential inhibitors from compound libraries utilizing a reverse genetics system developed for lineage I WNV cDNA clone and lineage I WNV replicon.

WNV subgenomic RNAs capable of replicating within cells (replicons) have been reported. (15, 17). The replicon RNA genome typically contains the 5′ Nontranslated region (5′ NTR), a portion of the Core coding region, a polyprotein encoding NS1 through NS5, and the 3′ NTR. Like replication of the WNV genome (reviewed in reference (4)), in the replicon cells, the viral RNA dependent RNA polymerase, NS5B, in conjunction with other viral nonstructural proteins and possibly cell factors (3), synthesize a minus strand RNA from the replicon subgenomic RNA template. The minus strand RNA in turn serves as templates for the synthesis of new genomic and message RNAs. Although data from studies of Kunjin virus suggest that both plus and minus strand RNA synthesis can occur in the absence of protein synthesis once the replication cycle establishes, viral protein synthesis is a prerequisite for replication of nascent RNAs (9). In addition to a selectable marker, neomycin phosphotransferaser gene, which is used for selection of the stable cell line, a luciferase reporter gene was also inserted into the RNA with its translation driven by the EMCV IRES (15). The expression of the reporter gene depends on the replication of the replicon RNA and can be easily monitored for identification of antiviral compounds (II).

For the purposes of drug screening it is preferable to use human epidemic-causing lineage I strains for assay setup to ensure that the identified compounds have a direct relevance to human disease.

Effective chemotherapeutics to treat WNV and other flaviviruses, known and emerging, are urgently needed. Although a limited number of inhibitors of flaviviruses have been identified, many of these have severe side effects, are not specific to flaviviruses, and are not known to be clinically effective and/or useful. For example, recent evidence suggested the use of nucleoside analogs as potential inhibitors of flaviviruses. Specific examples include inhibitors of orotidine monophosphate decarboxylase, inosine monophosphate dehydrogenase, and CTP synthetase. Although it appeared that these inhibitors may have been effective in virus infected Vero cells, their effectiveness in humans or animals (i.e., in vivo) is not known. Additionally, as these nucleoside analogs are broad-spectrum inhibitors of purine and pyrimidine biosynthesis, the occurrence of side effects and lack of flaviviral specificity would further limit their usefulness in a clinical setting.

Another nucleoside analog, the drug Ribavirin, was found to have some activity against WNV in vitro when administered in combination with interferon alpha-2b. However, the drug combination has not been shown to be effective in humans. Similarly, inhibitors to other protein activities of the viral genome, such as the helicase and protease activities encoded by NS3, have been explored; however, their clinical significance is unknown since their anti-WNV activities have not been tested in vivo. Finally, inhibitors of viral glycoprotein processing have been studied, but the prevalence of side effects due to inhibition of N-linked glycosylation, as well as difficulty in achieving therapeutic serum concentration levels, limit the usefulness of this type of compound. Thus, although there are a small number of known inhibitors for flaviviruses, none have been shown to be effective in humans. Accordingly, novel anti-flavivirus chemotherapies and/or improvements in the effectiveness, specificity, and clinical utility of known flavivirus chemotherapies are needed.

U.S. Application Publication 2006/0040958 to Guzi et al. describes pyrazolo[1,5-a]pyrimidine compounds as inhibitors of cyclin dependent kinases amd methods of making such compounds. Various pyrazolopyrimidines amd methods of making thereof are known in the art. WO92/18504, WO02/50079, WO95/35298, WO02/40485, EP94304104.6, EP0628559 (equivalent to U.S. Pat. Nos. 5,602,136, 5,602,137 and 5,571,813), U.S. Pat. No. 6,383,790, Chem. Pharm. Bull., (1999) 47 928, J. Med. Chem., (1977) 20, 296, J. Med. Chem., (1976) 19 517 and Chem. Pharm. Bull., (1962) 10 620 disclose various pyrazolopyrimidines.

WO01/92282 to Sommadossi et al. describes nucleosides for the treatment of a host infected with a flavivirus or pestivirus infection.

U.S. Pat. No. 6,812,219 to LaColla describes methods and compositions for treating flaviviruses and pestiviruses based on nucleosides.

Despite the current developments, there is a need in the art for novel flavivirus replication inhibitors, particularly, non-nucleoside based compounds.

All references cited herein are incorporated herein by reference in their entireties.

### BRIEF SUMMARY OF THE INVENTION

The present invention provides novel flavivirus replication inhibitors, pharmaceutical composition comprising one or more such flavivirus replication inhibitors and methods of treatment, prevention, inhibition or amelioration of one or more diseases associated with flavivirus replication using such inhibitors or pharmaceutical compositions. The replicon system used to discover flavivirus replication inhibitors of the invention is derived from a linkage I WNV isolate that have caused human infection and thus very relevant for finding an inhibitor to treat WNV infection.

In one aspect, the present invention relates to a flavivirus replication inhibitor, or pharmaceutically acceptable salts, solvates, esters or prodrugs of flavivirus replication inhibitor, wherein the flavivirus replication inhibitor is a compound of Formula (I)

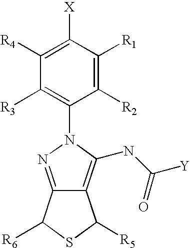

wherein X, Y and R₁₋₆ are members selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH, and pharmaceutically acceptable salts, solvates, esters or prodrugs thereof.

In a preferred embodiment, the flavivirus replication inhibitor is an inhibitor of West Nile virus replication. Preferably, the flavivirus replication inhibitor has an anti-viral activity tested in a live virus assay with 50% inhibitory concentration (IC50) less than 5 μM and 50% cytotoxic concentration (CC 50) at greater than 50 μM.

In certain embodiments, the flavivirus replication inhibitor is a compound having Formula (XIII) (also referred to herein as 18-B3)

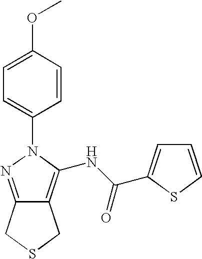

and derivatives thereof and pharmaceutically acceptable salts, solvates, esters or prodrugs thereof.

In certain embodiments, the flavivirus replication inhibitor is a compound of Formula (XIV) (also referred to herein as 18-D2)

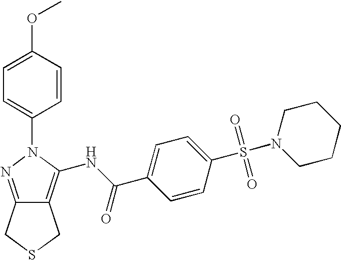

and derivatives thereof and pharmaceutically acceptable salts, solvates, esters or prodrugs thereof.

In certain embodiments, the flavivirus replication inhibitor is a compound of Formula (XV) (also referred to herein as 18-H5)

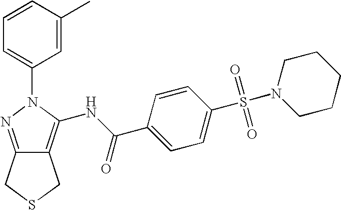

and derivatives thereof and pharmaceutically acceptable salts, solvates, esters or prodrugs thereof.

In certain embodiments, the flavivirus replication inhibitor is a compound of Formula (XVI) (also referred to herein as 20-E7)

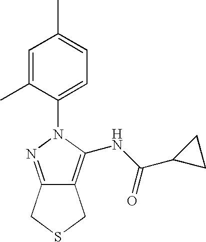

and derivatives thereof and pharmaceutically acceptable salts, solvates, esters or prodrugs thereof.

In certain embodiments, the flavivirus replication inhibitor is a compound of Formula (XVII) (also referred to herein as 253-B10)

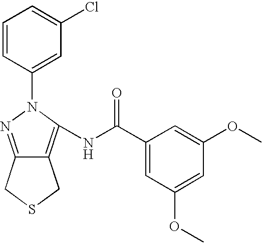

and derivatives thereof and pharmaceutically acceptable salts, solvates, esters or prodrugs thereof.

In certain embodiments, the flavivirus replication inhibitor is a compound of Formula (XVIII) (also referred to herein as 253-F8)

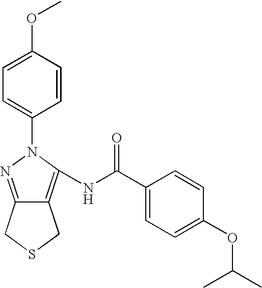

and derivatives thereof and pharmaceutically acceptable salts, solvates, esters or prodrugs thereof.

In certain embodiments, the flavivirus replication inhibitor is a compound of Formula (XIX) (also referred to herein as 253-F11)

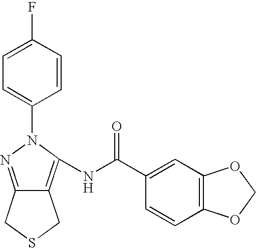

and derivatives thereof and pharmaceutically acceptable salts, solvates, esters or prodrugs thereof.

In certain embodiments, the flavivirus replication inhibitor is a compound 253-G8 of Formula (XX) (also referred to herein as 253-G8)

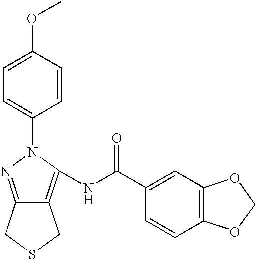

and derivatives thereof and pharmaceutically acceptable salts, solvates, esters or prodrugs thereof.

In certain embodiments, the flavivirus replication inhibitor is a compound 253-H8 of Formula (XXI) (also referred to herein as 253-H8)

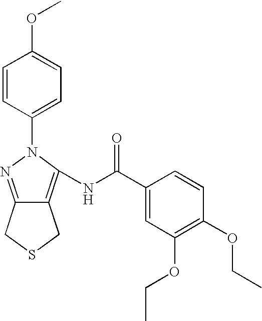

and derivatives thereof and pharmaceutically acceptable salts, solvates, esters or prodrugs thereof.

In another aspect, the present invention relates to a pharmaceutical composition comprising a pharmaceutically acceptable carrier and the compound of Formula (I).

In another aspect, the present invention relates to a process for making a pharmaceutical composition comprising combining the compound of Formula (I) and a pharmaceutically acceptable carrier.

In another aspect, the present invention relates to a method of treatment, prevention, inhibition or amelioration of one or more diseases associated with flavivirus replication in a subject in need thereof comprising administering to the subject a therapeutically effective amount of a pharmaceutical composition comprising administering to the subject a therapeutically effective amount of a pharmaceutical composition comprising the compound of Formula (I) and a pharmaceutically acceptable carrier. In certain embodiments of the method, the flavivirus caused disorder is a disorder related to a West Nile virus caused disorder.

In yet another aspect, the present invention relates to a flavivirus replication inhibitor, wherein the flavivirus replication inhibitor is a compound of Formula (II)

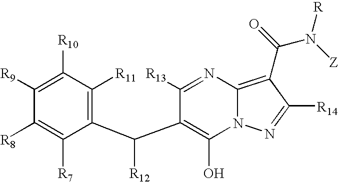

wherein R is a member selected from the group consisting of H, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH;

R₇ is a member selected from the group consisting of H, halogen, alkyl, aryl, alkoxy, arylalkyl, cycloalkyl, heterocyclyl, heterocyclylalkyl, heteroaryl, and heteroaryl alkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH; and

R₈₋₁₄ are members selected from the group consisting of H, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH. In a preferred embodiment, the flavivirus replication inhibitor is an inhibitor of West Nile virus replication. Preferably, the flavivirus replication inhibitor has an anti-viral activity tested in a live virus assay with 50% inhibitory concentration (IC50) less than 5 μM and 50% cytotoxic concentration (CC 50) at greater than 50 μM.

In a preferred embodiment, the flavivirus replication inhibitor is a compound 309-F6 of Formula (XI) (also referred to herein as 309-F6)

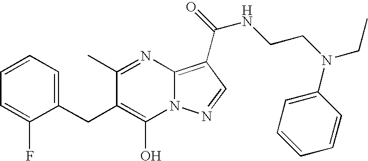

and derivatives thereof and pharmaceutically acceptable salts, solvates, esters or prodrugs thereof.

In certain embodiments, the flavivirus replication inhibitor is a compound of Formula (XII) (also referred to herein as 275-F9)

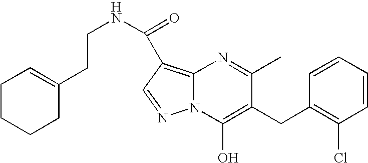

and derivatives thereof and pharmaceutically acceptable salts, solvates, esters or prodrugs thereof.

In certain embodiments, the flavivirus replication inhibitor is a compound of Formula (XXII) (also referred to herein as 310-B3)

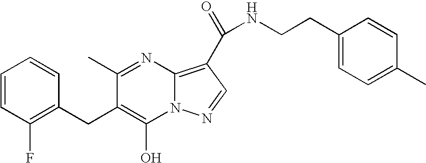

and derivatives thereof and pharmaceutically acceptable salts, solvates, esters or prodrugs thereof.

In another aspect, the present invention relates to a pharmaceutical composition comprising a pharmaceutically acceptable carrier and the compound of Formula (II).

In certain embodiments, the pharmaceutical composition comprises the pharmaceutically acceptable carrier and the compound of Formula (XI), derivatives thereof and pharmaceutically acceptable salts, solvates, esters or prodrugs thereof.

In another aspect, the present invention relates to a process for making a pharmaceutical composition comprising combining the compound of Formula (II) and a pharmaceutically acceptable carrier.

In another aspect, the present invention relates to a method of treatment, prevention, inhibition or amelioration of one or more diseases associated with flavivirus replication in a subject in need thereof comprising administering to the subject a therapeutically effective amount of a pharmaceutical composition comprising the compound of Formula (II) and a pharmaceutically acceptable carrier.

In yet another aspect, the present invention relates to a flavivirus replication inhibitor, wherein the flavivirus replication inhibitor is a compound of Formula (III)

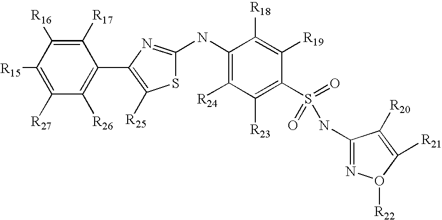

wherein R₁₅₋₂₇ are members selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH. In a preferred embodiment, the flavivirus replication inhibitor is an inhibitor of West Nile virus replication. Preferably, the flavivirus replication inhibitor has an anti-viral activity tested in a live virus assay with 50% inhibitory concentration (IC50) less than 5 μM and 50% cytotoxic concentration (CC 50) at greater than 50 μM.

In certain embodiments, the flavivirus replication inhibitor is a compound of Formula (IV) (also referred to herein as 101-G7)

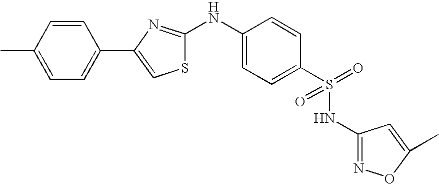

and derivatives thereof and pharmaceutically acceptable salts, solvates, esters or prodrugs thereof.

In another aspect, the present invention relates to a pharmaceutical composition comprising a pharmaceutically acceptable carrier and the compound of Formula (III). In a preferred embodiment, the pharmaceutical composition comprises the pharmaceutically acceptable carrier the compound of Formula (IV), derivatives thereof and pharmaceutically acceptable salts, solvates, esters or prodrugs thereof.

In another aspect, the present invention relates to a process for making a pharmaceutical composition comprising combining the compound of Formula (III) and a pharmaceutically acceptable carrier.

In another aspect, the present invention relates to a method of treatment, prevention, inhibition or amelioration of one or more diseases associated with flavivirus replication in a subject in need thereof comprising administering to the subject a therapeutically effective amount of a pharmaceutical composition comprising administering to the subject a therapeutically effective amount of a pharmaceutical composition comprising the compound of Formula (III) and a pharmaceutically acceptable carrier. In certain embodiments of the method the flavivirus caused disorder is a disorder related to a West Nile virus caused disorder.

In yet another aspect, the present invention relates to a flavivirus replication inhibitor, wherein the flavivirus replication inhibitor is at least one of

(a) a compound of Formula (V) (also referred to herein as 2-H7)

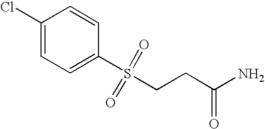

and derivatives thereof and pharmaceutically acceptable salts, solvates, esters or prodrugs thereof;

(b) a compound of Formula (VI) (also referred to herein as 24-C10)

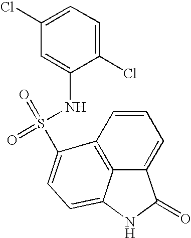

and derivatives thereof and pharmaceutically acceptable salts, solvates, esters or prodrugs thereof;

(c) a compound of Formula (VII) (also referred to herein as 42-E5)

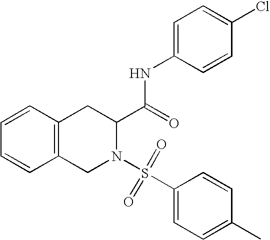

and derivatives thereof and pharmaceutically acceptable salts, solvates, esters or prodrugs thereof;

(d) a compound of Formula (VIII) (also referred to herein as 50-A8)

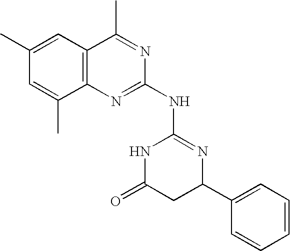

and derivatives thereof and pharmaceutically acceptable salts, solvates, esters or prodrugs thereof;

(e) a compound of Formula (IX) (also referred to herein as 63-C10)

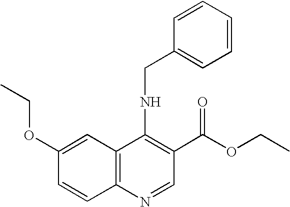

and derivatives thereof and pharmaceutically acceptable salts, solvates, esters or prodrugs thereof;

(f) a compound of Formula (X) (also referred to herein as 182-C2)

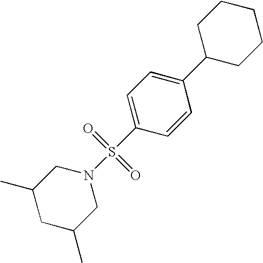

and derivatives thereof and pharmaceutically acceptable salts, solvates, esters or prodrugs thereof; or

(g) a compound of Formula (XXIII) (also referred to herein as 309-F6)

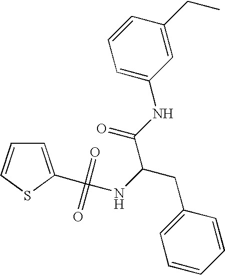

and derivatives thereof and pharmaceutically acceptable salts, solvates, esters or prodrugs thereof.

### BRIEF DESCRIPTION OF SEVERAL VIEWS OF THE DRAWINGS

The invention will be described in conjunction with the following drawings in which like reference numerals designate like elements and wherein:

FIG. 1A is a schematic representation of the WNV replicon used in the high-throughput screening (HTS) assay. The WNV and the subgenomic replicon genomes are depicted with the genes (Non-Structural gene NS1, 2A, 2B, 3, 4A, 4B and 5) shown as boxes. The WNV structural genes ((Core (C), Membrane (M) and Envelope (E)) were deleted in the replicons. Luciferase gene (Luc) and the Ned gene under the control of EMCV IRES were inserted at the 3′ NTR of the replicon.

FIG. 1B is a titration curve of the replicon cells BHK 26.5 in 96 well plates demonstrating linear relationship between cell number and luciferase activity.

FIG. 2 is a flow chart of the high-throughput screen analysis.

FIG. 3A is a graph demonstrating parameters of an in vitro luciferase enzyme assay to assess the compounds' inhibitory activity against luciferase enzyme. In two separate experiments, compounds were either tested with 3, 5, 10 μM or only 10 μM concentrations. The luciferase enzyme cell lysates were incubated with the indicated compounds for 30 minutes before luciferase substrate was added and the luminescence was measured. 1% DMSO was used as controls.

FIG. 3B is a structure of 52-F2, a compound that inhibited luciferase enzyme activity.

FIG. 3C is a picture of a Western blot gel showing that 52-F2 did not inhibit WNV viral protein level in the WNV replicon cells. 4×10⁴ cells of BHK26.5 cells in 24 well plates were treated with the indicated amount of 52-F2 for 48 hours. Total cell proteins from the treated cells were separated on SDS-PAGE and transferred to PVDF membrane. The blot was probed with a monoclonal antibody against WNV proteins. The same blot was probed for beta-actin for loading controls.

FIG. 4 is graph of IC50 determination for the “hit” compounds. 1×10⁴ BHK 26.5 cells in 96 wells were plated 24 hours before being treated with various concentrations of the indicated compounds. After 24 hours, the luciferase activity in the cells was quantified by using STEADY GLO reagent. On the y axis, the relative luminescence signal was shown with 1% DMSO treated cells as 100%. On the X axis is the concentration of the compounds in log scale.

FIG. 5 shows pictures of Western blot gels of the WNV protein in the compound treated WNV replicon cells. Mycophenolic acid (MPA) was used as positive control in the experiment. 4×10⁴ cells were seeded in 24 well plates 24 hours before the various concentrations of compounds were added. After 48 hours of drug treatment, SDS-Sample buffer were added to the wells to lyse the cells. The protein samples were separated on SDS-PAGE and transferred to PVDF membrane. Viral protein was detected by probing with a monoclonal antibody against WNV. The same membrane was stripped and probed for beta-actin with the signals shown at the bottom of the blot.

FIG. 6 is a picture of a Northern blot gel of compound treated WNV replicon cells. 4×10⁴ cells were seeded in 24 well plates. After 24 hours, the cells were treated with DMSO (lanes 2, 3) or the non-specific inhibitors, MPA (lane 1) or the compounds 275-F6 (lane 4), 18-B3 (lane 5) or 52-F2 (lane 6). After 48 hours, total RNA were isolated, separated on denaturing agrose gels and transferred to nylon membrane. Total RNA from a non-replicon cell (Huh-7 cell) was loaded in lane 7 as negative control. The membrane was probed with [³²P]-dCTP-labeled WNV NS5 and human beta-actin. The signal was quantified using a BioRad phosphoimager and WNV RNA level was calculated using beta-actin as a reference.

FIG. 7 is a structure of a compound 18-B3 of Table 3.

FIG. 8 is a structure of a compound 18-D2 of Table 3.

FIG. 9 is a structure of a compound 18-H5 of Table 3.

FIG. 10 is a structure of a compound 20-E7 of Table 3.

FIG. 11 is a structure of a compound 253-B10 of Table 3.

FIG. 12 is a structure of a compound 253-F8 of Table 3.

FIG. 13 is a structure of a compound 253-F11 of Table 3.

FIG. 14 is a structure of a compound 253-G8 of Table 3.

FIG. 15 is a structure of a compound 253-H8 of Table 3.

FIG. 16 is a structure of a compound 2-H7 of Table 3.

FIG. 17 is a structure of a compound 24-C10 of Table 3.

FIG. 18 is a structure of a compound 42-E5 of Table 3.

FIG. 19 is a structure of a compound 50-A8 of Table 3.

FIG. 20 is a structure of a compound 63-C10 of Table 3.

FIG. 21 is a structure of a compound 101-G7 of Table 3.

FIG. 22 is a structure of a compound 182-C2 of Table 3.

FIG. 23 is a structure of a compound 207-E5 of Table 3.

FIG. 24 is a structure of a compound 275-D9 of Table 3.

FIG. 25 is a structure of a compound 275-F9 of Table 3.

FIG. 26 is a structure of a compound 309-F6 of Table 3.

FIG. 27 is a structure of a compound 310-B3 of Table 3.

FIG. 28 is a structure of a compound 331-D11 of Table 3.

FIG. 29 is a structure of a compound 331-E11 of Table 3.

FIG. 30 is a graph demonstrating the activity of 101-G7 which has been chemically re-synthesized and re-tested in both the replicon assay and a live virus assay against an infectious WNV virus in tissue culture. The compound was added to WNV replicon cells as described at various concentrations. At 24 and 48 hours the luciferase activity was measured as a measurement of WNV replicon copy number. The luciferase activity was plotted against the concentration of the compound. An MTT assay using 3-(4,5-Dimethylthiazol-2-yl)-2,5-diphenyltetrazolium bromide was performed at the same time to measure the toxicity of the compound and plotted as % control.

FIG. 31 is a graph demonstrating that the 309-F6 compound (FIG. 26) inhibited WNV production. The antiviral assay for the 309-F6 compounds was conducted on BHK cells. The virus produced in the presence of 1% DMSO or various concentrations of compounds was tittered on Vero cells. The amount of virus relative to the 1% DMSO control was plotted on the y-axis; the concentration of the compound 309F6 is shown in μM on the x-axis. Data from two independently treated wells (Test 1 and Test 2) are shown.

### DETAILED DESCRIPTION OF PREFERRED EMBODIMENTS OF THE INVENTION

The invention provides novel flavivirus replication inhibitors of WNV other emerging flaviviruses, such as, for example, JEV, SLEV, AV, KV, JV, CV, YV, TBEV, DENV-1, DENV-2, DENV-3, DENV-4, YFV and MVEV, pharmaceutical composition comprising one or more such flavivirus replication inhibitors and methods of treatment, prevention, inhibition or amelioration of one or more diseases associated with flavivirus replication administering such inhibitors or pharmaceutical compositions.

Several classes of small molecule inhibitors of WNV replicon were discovered through high-throughput screening of a collection of more than 35, 000 compounds.

The invention will be described using 23 compounds as examples which were found to meet the criteria of selective inhibition of WNV replication and/or protein accumulation (Table 3) based on IC50 at most 10 uM and CC50 at least 10 uM.

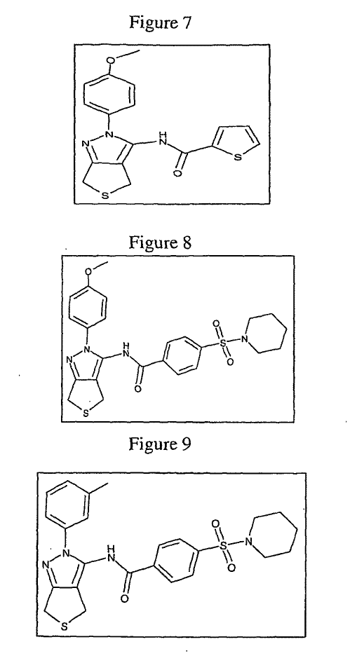

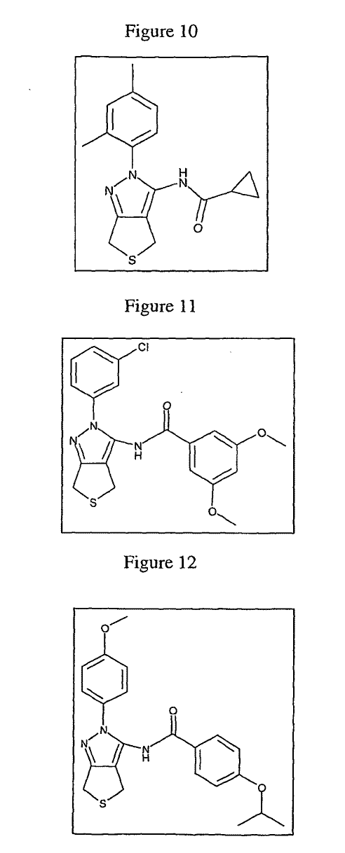

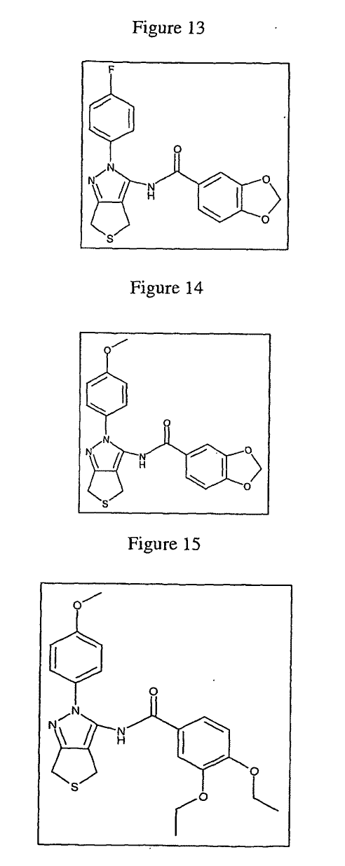

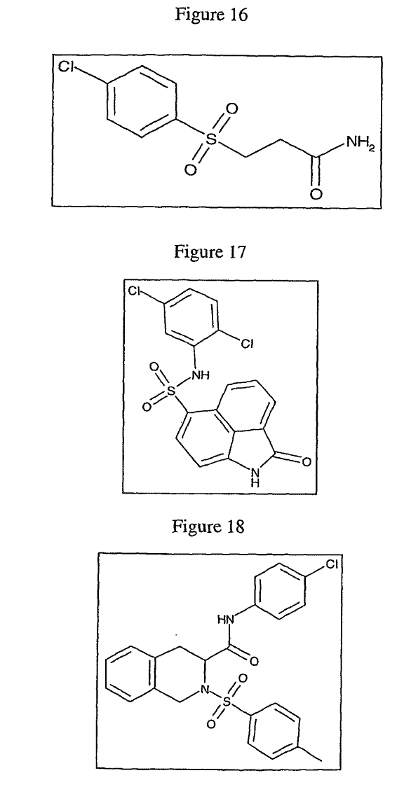

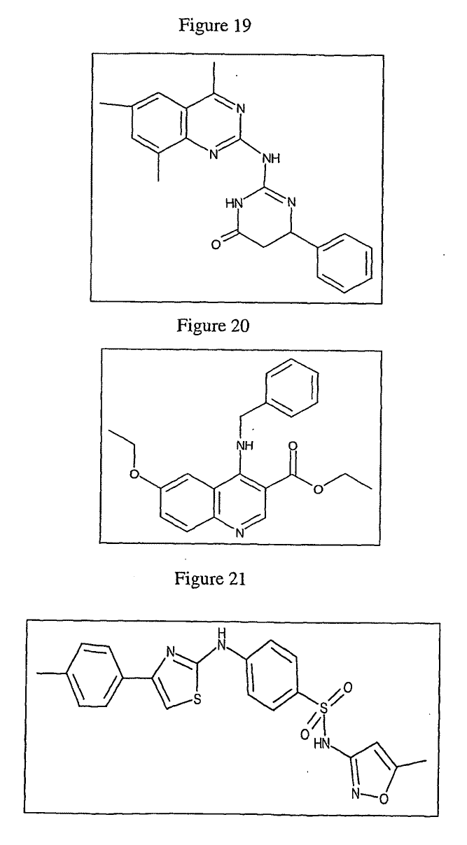

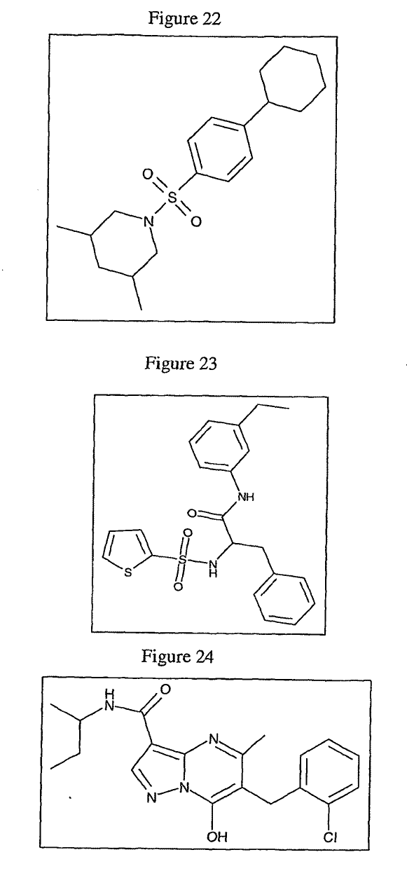

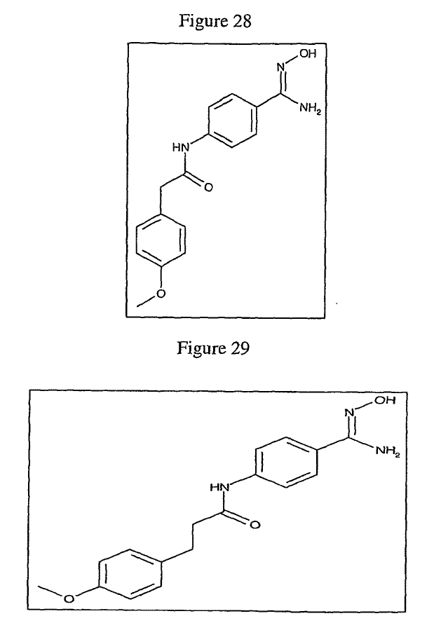

As summarized in Table 3 below and FIGS. 7-29, some of the compounds share common structural features. Based on the chemical structure, the 23 compounds can be grouped into 11 different classes. Eight compounds were single hit compounds that share no similarity with other hits. Among these, 24-C10 (FIG. 17), 42-E5 (FIG. 18), 50-A8 (FIG. 19), 63-C10 (FIG. 20), 101-G7 (FIG. 21), and 207-E5 (FIG. 23) are chemically attractive. However, 2417 (FIG. 28) and 182-C2 (FIG. 29) are less desirable because they have simple structures that lack uniqueness and practically accessible functional groups for further modification.

Compounds 331-D11 (FIG. 28) and 331-E11 (FIG. 29) have similar chemical structures, but their hydroxyamidine group is potentially reactive and may have potential stability issues. Furthermore, these two compounds are highly polar and are problematic in pharmacokinetics. Therefore, these two compounds are less attractive for further development.

A preferred group of flavivirus replication inhibitors of the invention consists of parazolotrahydrothophenes (PyrozoloHTH). PyrozoloHTH compounds have a pyrozolotetrahydrophene core structure with modification at the aryl phenol ring group and the 5-position amide group. PyrozoloHTH compounds are represented by Formula (I)

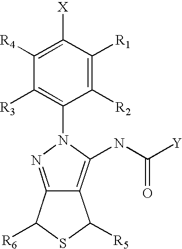

wherein X, Y and R₁₋₆ are members selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH.

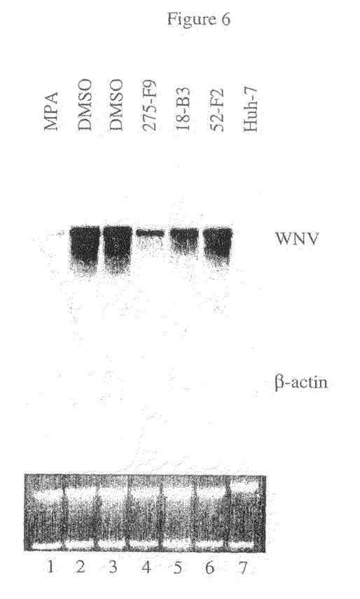

Exemplary non-limiting compounds include 18-B3, 18-D2, 18-H5, 20-E7, 253-B10, 253-F8, 253-F11, 253-G8, and 253-H8 (FIGS. 7-15) (Tables 1 and 3). The 1050 of these compounds ranged from 2 to 10 uM in the replicon cell. In addition, a few similar compounds exist in the inventors' library that are not active (not shown), indicating that there is a preliminary structure-activity relationship (SAR). One representative compound, 18-B3 (FIG. 7), clearly inhibited the WNV RNA replication at 10 μM (FIG. 6), indicating the authenticity of this class of compounds.

TABLE 1

| | | For- |
|---|---|---|
| Com- | | mula |
| pound | Structural Formula | No. |
| 18-B3 | 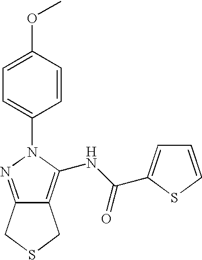 | (XIII) |
| 18-D2 | 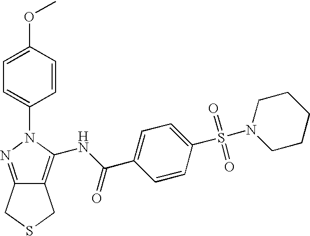 | (XIV) |
| 18-H5 | 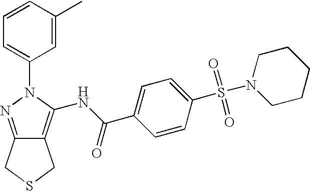 | (XV) |
| 20-E7 | 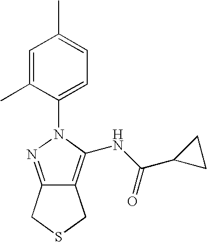 | (XVI) |
| 253-B10 | 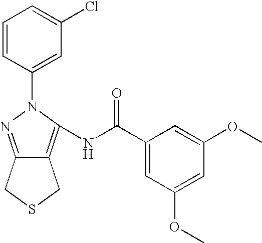 | (XVII) |
| 253-F8 | 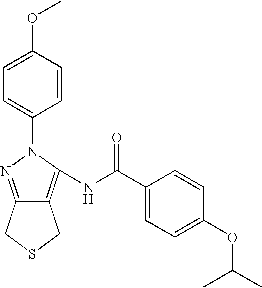 | (XVIII) |
| 253-F11 | 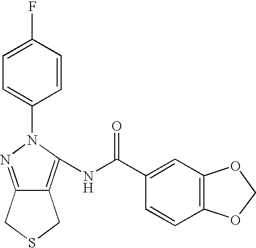 | (XIX) |
| 253-G8 | 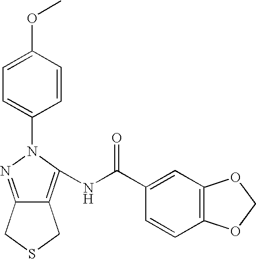 | (XX) |
| 253-H8 | 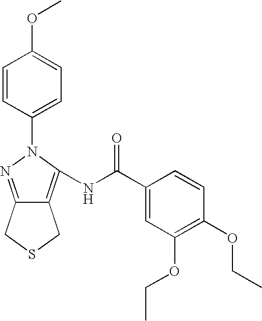 | (XXI) |

Another preferred group of the flavivirus replication inhibitors of the invention consists of pyrazolopyrimidines. Pyrazolopyrimidine compounds are represented by Formula (II)

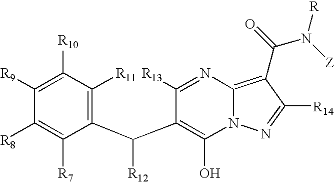

wherein R is a member selected from the group consisting of H, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH;

R₇ is a member selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclyl alkyl, heteroaryl, heteroarylalkyl, CF₃, OCF₃, CN, and —OH; and

R₈₋₁₄ are members selected from the group consisting of H, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH. In a preferred embodiment, the flavivirus replication inhibitor is an inhibitor of West Nile virus replication. Preferably, the flavivirus replication inhibitor has an anti-viral activity tested in a live virus assay with 50% inhibitory concentration (IC50) less than 5 μM and 50% cytotoxic concentration (CC 50) at greater than 50 μM.

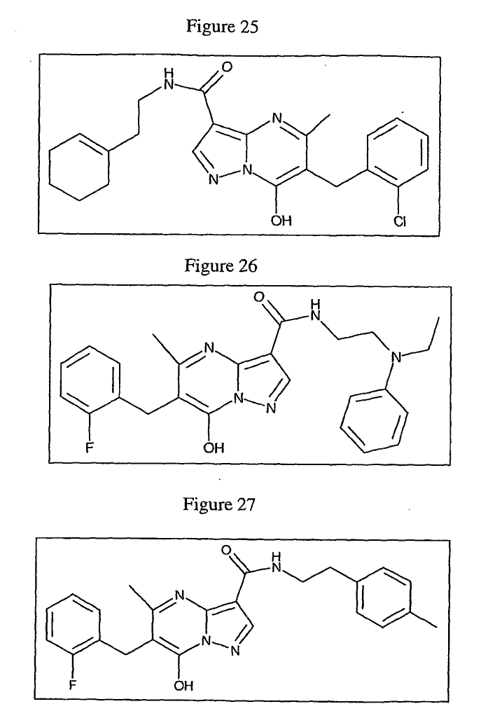

Exemplary pyrazolopyrimidine compounds are 275-D9, 275-F9, 309-F6 and 310-B3 (FIGS. 24-27). These compounds have a benzyl group with an electron withdrawing constituent between a methyl and hydroxy group. An amide group with varying substitutions is present at the same position. One of the pyrazolopyrimidine compound (309-E6, not shown) has only a single atom differences from 310-B3, yet it had no activity in the Western blot analysis, demonstrating a potential SAR at the benzyl group for this class of compounds. Although the pyrazolopyrimidine compounds are generally less potent than the PyrozoloHTH compounds in the assays performed, the chemistry is attractive and one of the compound, 275-F9 (FIG. 25) was clearly capable of reducing the WNV RNA level in the northern blot analysis (FIG. 6).

In a preferred embodiment, the flavivirus replication inhibitor is a compound 309-F6 of Formula (XI) (also referred to herein as 309-F6)

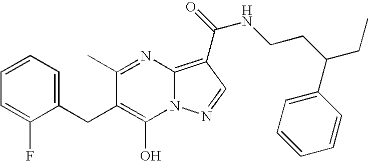

and derivatives thereof and pharmaceutically acceptable salts, solvates, esters or prodrugs thereof.

In certain embodiments, the flavivirus replication inhibitor is a compound of Formula (XII) (also referred to herein as 275-F9)

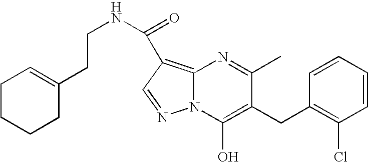

and derivatives thereof and pharmaceutically acceptable salts, solvates, esters or prodrugs thereof.

In certain embodiments, the flavivirus replication inhibitor is a compound of Formula (XXII) (also referred to herein as 310-B3)

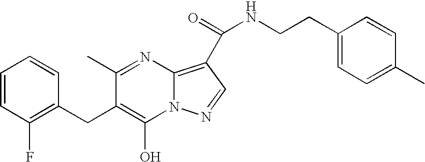

and derivatives thereof and pharmaceutically acceptable salts, solvates, esters or prodrugs thereof.

In yet another aspect, the present invention relates to a flavivirus replication inhibitor, wherein the flavivirus replication inhibitor is a compound of Formula (III)

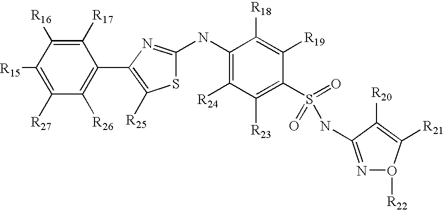

wherein R₁₅₋₂₇ are members selected from the group consisting of H, halogen, alkyl, aryl, alkoxy, arylalkyl, cycloalkyl, heterocyclyl, heterocyclylalkyl, heteroaryl, and heteroaryl alkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH. In a preferred embodiment, the flavivirus replication inhibitor is an inhibitor of West Nile virus replication. Preferably, the flavivirus replication inhibitor has an anti-viral activity tested in a live virus assay with 50% inhibitory concentration (IC50) less than 5 μM and 50% cytotoxic concentration (CC 50) at greater than 50 μM.

In certain embodiments, the flavivirus replication inhibitor is a compound of Formula (IV) (also referred to herein as 101-G7)

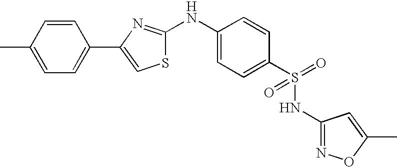

and derivatives thereof and pharmaceutically acceptable salts, solvates, esters or prodrugs thereof.

In yet another aspect, the present invention relates to a flavivirus replication inhibitor, wherein the flavivirus replication inhibitor is at least one of

(a) a compound of Formula (V) (also referred to herein as 2-H7)

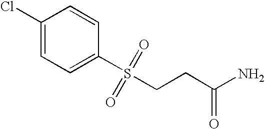

and derivatives thereof and pharmaceutically acceptable salts, solvates, esters or prodrugs thereof;

(b) a component of a structure (VI) (also referred to herein as 24-C10)

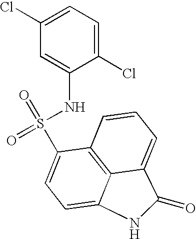

and derivatives thereof and pharmaceutically acceptable salts, solvates, esters or prodrugs thereof;

(c) a compound of Formula (VII) (also referred to herein as 42-E5)

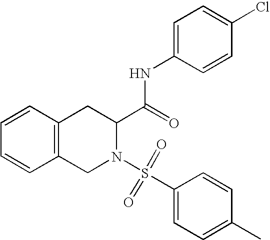

and derivatives thereof and pharmaceutically acceptable salts, solvates, esters or prodrugs thereof;

(d) a compound of Formula (VIII) (also referred to herein as 50-A8)

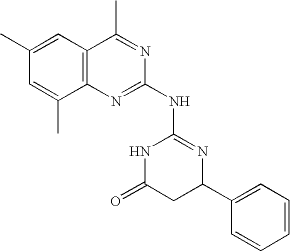

and derivatives thereof and pharmaceutically acceptable salts, solvates, esters or prodrugs thereof;

(e) a compound of Formula (IX) (also referred to herein as 63-C10)

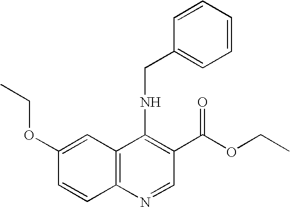

and derivatives thereof and pharmaceutically acceptable salts, solvates, esters or prodrugs thereof;

(f) a compound of Formula (X) (also referred to herein as 182-C2)

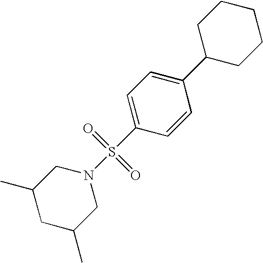

and derivatives thereof and pharmaceutically acceptable salts, solvates, esters or prodrugs thereof; or

(g) a compound of Formula (XXIII) (also referred to herein as 309-F6)

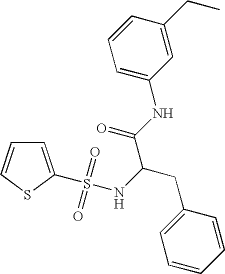

and derivatives thereof and pharmaceutically acceptable salts, solvates, esters or prodrugs thereof.

Pharmaceutical Compositions

The compounds will be studied for their pharmacology kinetics and for their toxicity profile in relevant animal models as described in publicly available literature.

Thus, in another aspect, the present invention relates to a pharmaceutical composition comprising a pharmaceutically acceptable carrier and one or more of the “active” compounds flavivirus replication inhibitors described herein (e.g., compounds of Formulas (I)-(XXIII)) including pharmaceutically acceptable salts, solvates, esters or prodrugs thereof.

The pharmaceutical compositions comprising the compositions of the invention may be in a variety of conventional depot forms. These include, for example, solid, semi-solid and liquid dosage forms, such as tablets, pills, powders, liquid solutions or suspensions, liposomes, capsules, suppositories, injectable and infusible solutions. The preferred form depends upon the intended mode of administration and prophylactic application.

Such dosage forms may include pharmaceutically acceptable carriers and adjuvants which are known to those of skill in the art. These carriers and adjuvants include, for example, RtBI, ISCOM, ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes such as protamine sulfate, disodium hydrogen phosphate, sodium chloride, zinc salts, colloidal silica, magnesium trisilicate, polyvinyl pyrrolidone, cellulose-based substances, and polyethylene glycol. Adjuvants for topical or gel base forms may be selected from the group consisting of sodium carboxymethylcellulose, polyacrylates, polyoxyethylene-polyoxypropylene-b-lock polymers, polyethylene glycol, and wood wax alcohols.

The compositions of the invention may also include other components or be subject to other treatments during preparation to enhance their bioavailability or to improve their tolerance in patients.

In another aspect, the present invention relates to a process for making a pharmaceutical composition comprising combining one or more of the “active” compounds flavivirus replication inhibitors described herein (e.g., compounds of Formulas (I)-(XXIII)) and a pharmaceutically acceptable carrier. The term “combining” includes all aspects of combining the components, including but not limited to dissolving, admixing, dispersing, embedding and encapsulating. The term “combining” encompasses partial mixing of the components and combining the components with additional components which would serve as a layer between components such that no or substantially no immediate contact between the active components is observed.

In another aspect, the present invention relates to a method of treatment, prevention, inhibition or amelioration of one or more diseases associated with flavivirus replication in a subject in need thereof comprising administering to the subject a therapeutically effective amount of a pharmaceutical composition comprising one or more of the “active” compounds flavivirus replication inhibitors described herein (e.g., compounds of Formulas (I)-(XXIII)) and a pharmaceutically acceptable carrier. treatment, prevention, inhibition or amelioration of one or more diseases associated with flavivirus replication including administering one or more such inhibitors or pharmaceutical compositions.

Any pharmaceutically acceptable dosage route, including parenteral, intravenous, intramuscular, intralesional or subcutaneous injection, may be used to administer the compositions of the invention. For example, the composition may be administered to the patient in any pharmaceutically acceptable dosage form including those which may be administered to a patient intravenously as bolus or by continued infusion over a period of hours, days, weeks or months, intramuscularly—including paravertebrally and periarticularly—subcutaneously, intracutaneously, intra-articularly, intrasynovially, intrathecally, intralesionally, periostally or by oral or topical routes. Preferably, the compositions of the invention are in the form of a unit dose and will usually be administered to the patient orally.

The pharmaceutical compositions comprising the compositions of the invention or derivatives or modifications thereof may also be administered to any animal, including, but not limited to, horses, cattle, monkeys, birds, dogs, cats, ferrets, rodents, squirrels, and rabbits to provide a protective immune response therein against a flavivirus, e.g., WNV, DENV, or any other known or emerging flavivirus, and/or to treat or limit the lateral passage of infection by a flavivirus to humans. For example, the pharmaceutical compositions comprising the compositions of the invention can be combined with animal feed stock and/or water provisions, dog food, cat food, bird food, or rodent food. One of skill in the art will understand this method of administration is sometimes referred to as “bait dropping,” in which the pharmaceutical composition is included within the food and/or water of the organism to be treated.

Screening Method

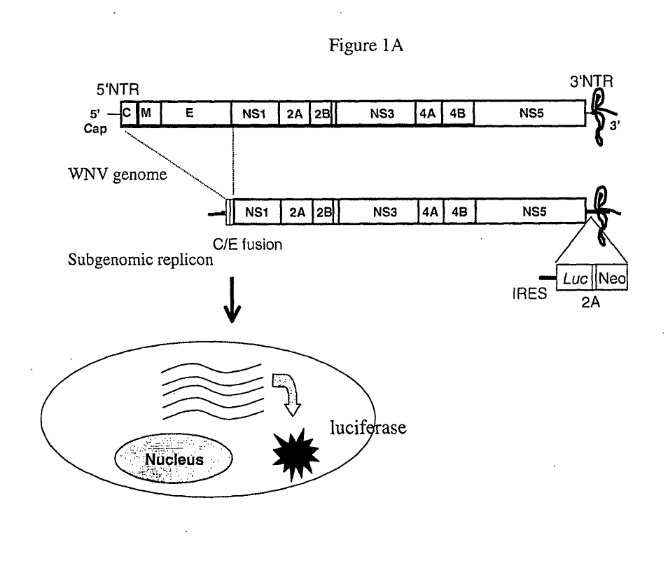

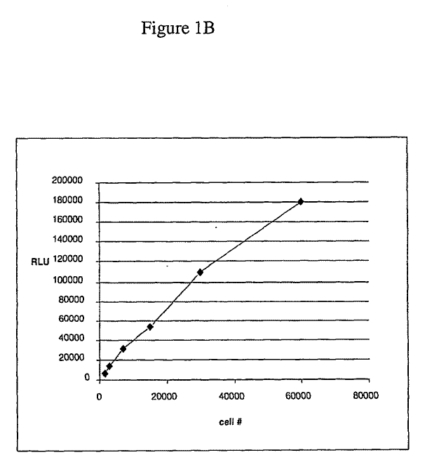

Ten micromolar concentrations of a selected compound were incubated with BHK 26.5 cells expressing luciferase whose activity is dependent upon WNV replicon replication (FIG. 1A). Primary “hits” were defined by the ability to reduce the amount of luciferase enzyme activity by more than 50%, compared to controls.

Primary “hits” were validated as selective for WNV (as opposed to inhibitory of luciferase) in a second assay, in which compound was tested for the ability to reduce the amount of WNV RNA and protein production.

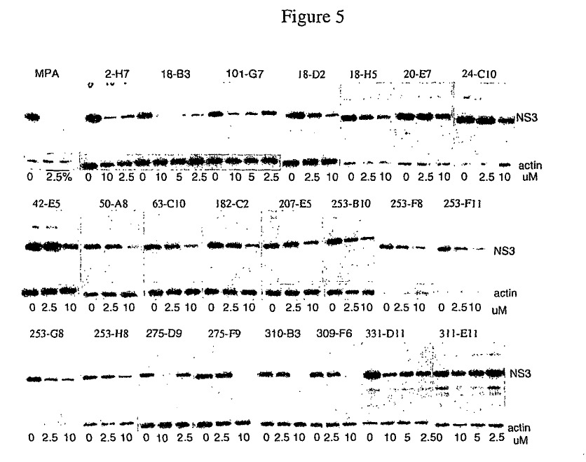

The mechanisms of action for these “hits” are being investigated. Whether the compounds are targeting a viral protein or a cellular process that is involved is not known. Inventors are developing virus resistance to the most potent “hits” in order to find out whether a viral protein in the WNV RNA replication complex is involved. Without being bound by a specific theory, based on the western blot data (FIG. 5), it is believed that none of the compounds inhibited the NS3 protease activity, since inhibition of NS3 will affect the WNV polyprotein processing and there is no visible change on the SDS PAGE. Inventors are establishing WNV polymerase and RNA unwinding helicase assays to investigate whether the compounds can inhibit these enzymatic activities in vitro.

Although the “hits” clearly demonstrate ability to inhibit the WNV replication-dependent luciferase activity and reduce WNV viral protein accumulation and viral RNA levels in replicon cells, the compounds need to be re-synthesized and re-tested. If the re-synthesized compounds do not inhibit, some contaminating chemical or modified products in the compound library must exist. In that case, an analysis should be performed to determine the nature of this component. Compound 101-G7 was re-synthesized and its activity was confirmed as described below.

Several small molecules, in particular, nucleoside analogs have been reported to be active in controlling flavivirus infections (13), supporting the contention that antiviral drugs could be useful in combating the emerging public health threat produced by WNV infection. The high throughput screen “hits” identified by inventors clearly indicate the potential of developing such small molecule non-nucleoside inhibitors. These hits, though not as potent, can serve as the starting point for rational design of more potent WNV inhibitors. Furthermore, the “hits” can be useful research tools in dissecting the molecular mechanisms of the WNV replication and viral host interaction.

High Throughput Assay

To find the best condition for a high throughput assay, among the many variables, cell density, DMSO concentration, and the effect of freeze/thaw cycle of cells on the assay were considered. First, BHK 26.5 cells were seeded 24 hours before the assay at a density ranging from 1500 to 30,000 cells per well. As shown in FIG. 1B, the luminance signal measured from these cells ranged from 5000 to 100,000 RLU (FIG. 1B). Since the signal correlated closely with cell number with a slope of approximately one at the lower cell density, a linear range density of 10,000 cell/well for the HTS assay which consistently produced easily measurable luminescence signals (30,000 RLU) was chosen. At this density the cells are not overly confluent, a condition in which replicon copy number might be adversely affected (14). Secondly, BHK 26.5 stock cells were frozen, thawed and cultured again before being used in the HTS assays. Luciferase activity did not change significantly over at least four cycles of freeze and thaw and over 10 passages (data not shown). For our assay, HTS was performed with cells that were within 4 passages of each other from the original frozen stock. Thirdly, the cells were tolerant to 1% DMSO, with a slight decrease in the luminance signal produced when 2% DMSO were used (data not shown). Therefore, DMSO was limited to 1% when compounds were added.

Minimal assay signal variation and consistent high signal to background ratio is key to the success of an HTS assay. Z', a statistical measurement of the distance between the standard deviations for the signal versus the noise of an assay was determined by treating multiple samples with mycophenolic acid (MPA), a known albeit nonspecific, WNV inhibitor. Briefly, 40 wells of a 96 well plate were treated with 2.5% MPA in 1% final DMSO, and 40 wells were treated with 1% DMSO only. After 24 hours, the luminescence was measured. The Z′ was calculated by the equation: 1−(3×SD_{DMSO}+3×SD_{MPA})/Mean_{DMSO}−Mean_{MPA}). The Z′ is approximately 0.5, indicating that the assay is reasonably reliable. The assay consistently produced a noise to background ration of around 8 to 1.

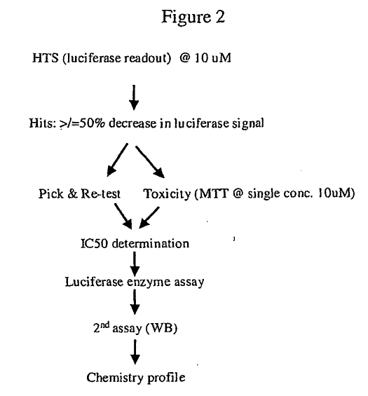

The HTS strategy follows the flowchart outlined in FIG. 2. 35,000 compounds were screened from a chemical library described below, using a cutoff of 50% reduction in luminescence readout as the criteria for further study. Upon completion of the screen, approximately 1100 compounds were selected, making the hit rate of 3%. This is considerably higher than expected, implying many false positive and/or cytotoxic compounds. Nevertheless, the compounds were picked out from the “daughter” plates and re-checked at a single 10 μM concentration. In addition, a duplicate 96 well plates were also plated and the toxicity of the compounds was determined in an MTT assay. Upon completion of these assays, most compounds were disqualified as either false positives or being toxicity to cells. As a result, only a small number of compounds were further analyzed directly from the “mother” plate stock. After IC50 determination and secondary assay to rule out luciferase inhibitors, 23 “hits” were identified. The structure and activity of these hit compounds are summarized in Table 3.

Eliminating Luciferase Enzyme Inhibitors

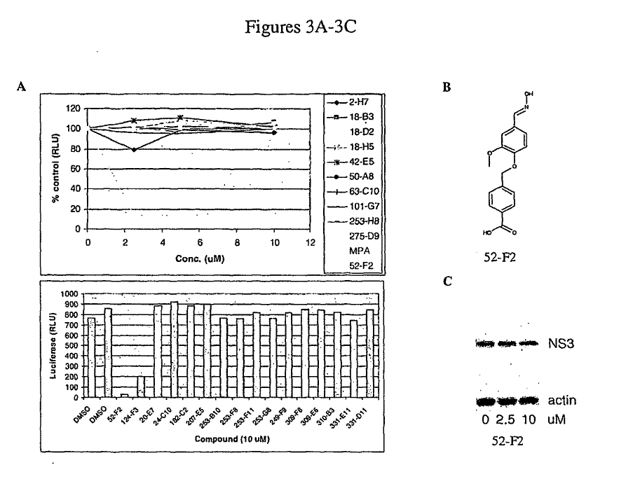

Although the initial HTS and the repeat assays have identified a number of compounds capable of reducing the luciferase activity produced in the WNV replicon BHK 26.5 cell line. It is conceivable that some of these inhibitors could have inhibited the luciferase enzyme itself rather than the WNV viral replication. To rule out this possibility, the identified compounds were tested in an in vitro luciferase enzyme assay by incubating the compounds with cell lysates prepared from the WNV replicon cell. Under such conditions, replication of WNV does not contribute to luciferase activity and inhibition of the luciferase enzyme can be identified. FIG. 3 shows the in vitro results. As expected, MPA and DMSO did not reduce the luciferase activity in this assay. However, the luminescence signal was dramatically reduced after incubation of the luciferase cell lysate with two hit compounds, 52-F2 and 124-F3 (structures not shown). The inhibition was not observed when the compounds were added immediately before luminescence reading, but after a 30-minute incubation of luciferin substrate and the cell lysate, indicating a direct inhibition of the luciferase enzyme by the compounds. The two compounds also inhibited luciferase lysates from a pGL-3 vector transfected cell line (data not shown). Consistent with these data, 52-F2 did not reduce WNV NS3 protein level even though the luciferase activity was inhibited more than 10 fold at 10 μM (FIGS. 3B, 3C). Thus the remaining HTS hits were not luciferase inhibitors, suggesting potential in inhibiting the viral replication mechanism.

IC50 Determination of the “Hit” Compounds

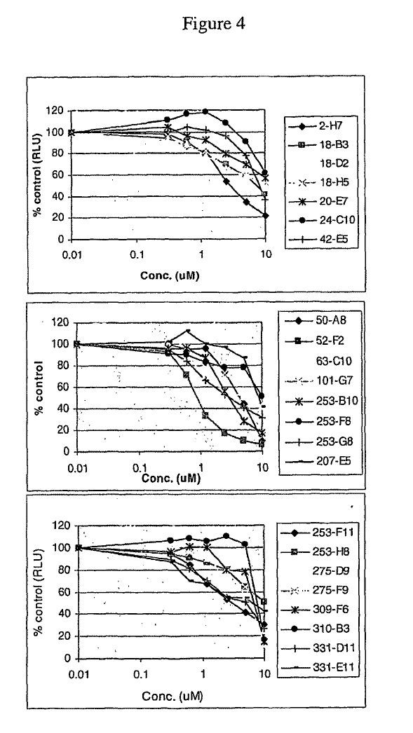

To quantify and rank the activity of the 23 remaining compound “hits”, a serial dilution of each compound was incubated with WNV replicon cells to determine the concentration that inhibits 50% of the luciferase readout (IC50s). FIG. 4 shows the IC50 curves of the “hits” including the luciferase inhibitor 52-F2. A number of compounds had significant effect only at the highest, 10 μM concentration. However, several compounds had IC50s between 2 and 5 (Table 3), which is very reasonable for primary HTS hits from a compound library. Only a few compounds could cause 90% inhibition in the replicon cells at the concentrations tested.

Confirmation of the WNV Inhibitor Compounds

Although it is reassuring that multiple tests have yielded similar positive results, the ability of the compounds to inhibit WNV replication was further assessed in secondary assays. In the western blot analysis shown in FIG. 5, total cell proteins were prepared from WNV replicon cells treated with the hit compounds and the viral protein NS3 was detected with a monoclonal activity against West Nile virus (15). The same blots were stripped and probed for beta-actin to show equal loading. As shown in FIG. 5, all the compounds had minimal effect on the beta-actin level, which is consistent with the MTT data showing that these compounds are not cytotoxic at the 10 uM concentration used. The control compound, MPA, reduced WNV NS3 protein level at the 2.5 ug/ml concentration used. The western blot data demonstrate that the tested compounds all reduced WNV NS3 protein level in the replicon cells after 48-hour drug treatment. A dose dependency is also clearly seen with several compounds, e.g. 18-D2 and 331-D11 (structures shown in FIGS. 7-29). However, a few compounds reduced viral protein only at the 10 uM, the highest concentration used. Examples include 310-B3 and 309-F6 (structures shown in Table 3). When compared with the IC50 determined for these compounds in the luciferase assay, there was a good correlation between the luciferase reduction and the viral protein reduction. These results clearly showed that the “hit” compounds are WNV inhibitors and that the luciferase readout is a good indicator for a true antiviral assays.

To confirm that the compounds can indeed inhibit WNV RNA replication, the WNV RNA level in the replicon cells treated with compounds by Northern blot analysis. Several hit compounds, 18-B3, 275-F9, 275-D9, 52-F2 and MPA were selected to treat the BHK 26.5 cells at 10 μM. FIG. 6 shows that after 48-hour treatment, WNV RNA was reduced more than five fold in cells treated with 2.5% MPA (FIG. 6, comparing lane 1 with lanes 2 and 3). The “hit” compounds 18-B3 and 275-F9 reduced WNV RNA by 1.5 and 2 fold, respectively (FIG. 6, lanes 4, 5). 18-B3 is one of the nine compounds that have similar structures while 275-F9 is one from another class of four compounds (Table 3). Consistent with its inability to reduce WNV protein level (FIG. 3C), 52-F2 did not reduce the WNV RNA level (FIG. 6, lane 6). These data indicate that the “hit” compounds inhibited WNV RNA replication. Together with the western blot results, the Northern blot data clearly demonstrate that true inhibitors of WNV replicon replication were likely identified.

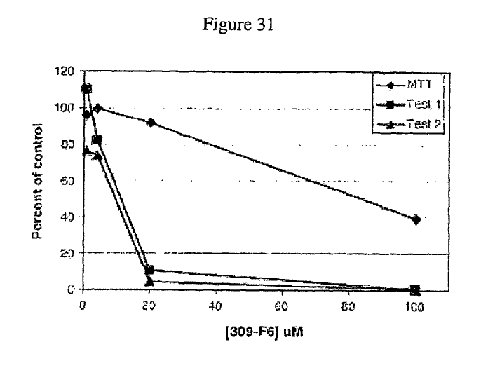

Although the compounds selected from the WNV replicon screen were shown to reduce replicon protein and RNA synthesis, the data from the Western and Northern blots also showed that these compounds had limited potency indicating that they might require further improvement by chemical modification to become potent virus inhibitors. Several of these original compounds selected from the WNV replicon based screen were evaluated in an antiviral assay against WNV live virus. FIG. 31 shows that one of the compounds (309-F6; a pyrazolopyrimidine) had good activity in this assay with the WNV titer being reduced by approximately 90% at 20 μM. In parallel toxicity assessment, only slight cell toxicity was observed at 20 μM. 309-F6 had an estimated selective index of more than 8. 275-D9 ((6-(2-Chloro-benzyl)-7-hydroxy-5-methyl-pyrazolo[1,5-a]pyrimidine-3-carboxylic acid sec-butylamide)) had no detectable activity in the WNV infection assay demonstrating SAR. Modifications of this compound would be further investigated

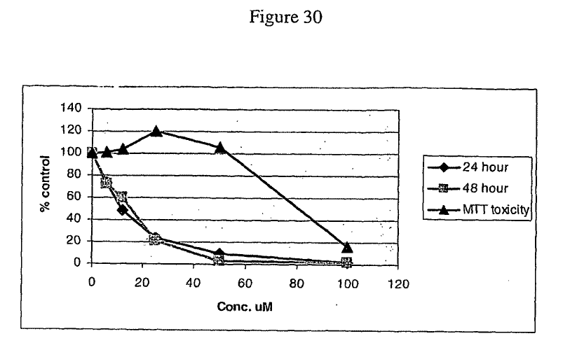

Additional confirmation was obtained for the compound 101-G7. Specifically, inventors re-synthesized 101-G7 through conventional chemistry. This is to ascertain that the activity detected from the compound library is due to the specified chemical rather than some contaminating or breakdown products present in the library. After 101-G7 was synthesized, it was re-tested in the WNV replicon cells and in the WNV infection assay. FIG. 30 show that the re-synthesized compound had good activity in reducing the luciferase activity of the WNV replicon cell after both 24 and 48 hour incubation with the replicon BHK cells. The toxicity of 101-G7 in these assays was about 80 μM, well above the IC50. When the re-synthesized compound was tested in the WNV infection assay, 101-G7 also retained activity. In three independent experiments, each experiment was done in duplicate, the re-synthesized 101-G7 had IC50 between 3 and 15 μM and CC50 >40 μM (the highest concentration used). Table 2 shows the IC50 and CC50 of 101-G7 in BHK cells for each of the experiment.

The toxicity of 101-G7 was also measured by MTT assay and CC50 was calculated. 40 μM is highest concentration used.

TABLE 2

| | | Tox |
|---|---|---|
| IC50 | IC50 | CC50 |
| (uM) | (uM) | (uM) |
| 15.5 | 13 | >40 |
| 7.9 | 3 | >40 |
| 6.5 | nd | >40 |

Cells and Media

The WNV sequence was an infectious strain derived from an immuncomprised patient. The cDNA and WNV replicon construction was described in Rossi et al. (15). The WNV replicon (FIG. 1) cell line BHK 26.5, derived from BHK cells (described by Rossi et al, 2005 and obtained from Peter W. Mason), was maintained in regular Modified Eagles Media (MEM) with 10% FBS, 1× penicillin/streptomycin/2.5 mg/ml Plasmocin (InvivoGen) and 400 ug/ml G418. Cells were passaged every 3 days at 1:20 to 1:40. For high throughput assay, cells were trypsinized, counted, and plated in 96 well plates in media with only 3% FBS to prevent cell over confluency and to reduce compound-protein binding. The cells used were all within 4 passages to ensure repeatability. The WNV sequence was an infectious strain derived from an immuncomprised patient. The cDNA and WNV replicon construction was described in Rossi et al. (15).

Chemical Library

The chemical library includes selected compounds from the collections of Asinex Inc. (Moscow, Russia), Chembridge Inc. (San Diego, Calif.) and Maybridge Inc. (Cornwall, UK). The compounds were selected by computational means for diversity, solubility and drug-like qualities, eliminating highly reactive groups and species known to exhibit non-specific biological effects, and exhibiting an average molecular weight of approximately 350 Daltons. The original compound library stock plates (“mother” plates) are comprised of wells containing compounds corresponding to 80 wells per 96 well plates, specifically columns 2 to 11, rows A-H. Each compound was dissolved in tissue-culture grade dimethylsulfoxide (DMSO) at an average concentration of 10 mM. Dilution (“daughter”) plates have been produced by replica-plating of the mother plates in DMSO to give an average concentration of 1 mM. Both mother and daughter plates were sealed with plastic film and stored at ±20° C.

Antiviral treatment of replicon-expressing cells and high throughput assay.

Replicon-harboring cells were plated 96-well plates at 10,000 cells per well in MEM plus 3% FBS. 24 hours after plating, compounds were added to the wells by a Robotic liquid handler (Biomek NX) to a final concentration of 10 μM and 1% DMSO. For each 96 well plate, A1 to A8 were left open, H1 to H4 contained 1% DMSO, H5-H8 contained Mycophenolic acid (Sigma-Aldrich, St. Louis, Mo.), which was included as positive controls. After 24 hours of treatment, the media were removed and the luciferase activity expressed in the replicon cells was quantified. When cyto-toxicity was measured, duplicate 96 well plates were plated with BHK 26.5 replicon cells. One plate was used for luciferase assay and one plate was used for measuring toxicity using MTT assay.

Luciferase Assay

Luciferase assay was performed by using the STEADY GLO reagent (Promega Corp. Madison, Wis.) according to the manufacturer's recommendation with slight modifications. Briefly, the culture media from the 96 wells were removed by dumping on paper towels.

100 μl of a 1:1 mixture of luciferase regent and culture media were added to the plate. After 5-minute incubation, the luminescence was read on a TOPCOUNT (PerkinElmer, Wellesley, Mass.).

Western Blot Analysis

Total cell lysates from replicon cells were harvested from replicon cells in 1×SDS sample buffer. The lysates were heated at 70° C. for 10 min in the presence of DTT before electrophoresis on a 10% Tris-glycine SDS polyacrylamide gel (Invitrogen) in 1× Tris-glycine buffer. The protein was transferred to PVDF (Invitrogen) membrane. Following the transfer, the membrane was rinsed once with TBS containing 0.5% Tween-20 (TBS-Tween) and blocked in TBS-Tween containing 5% non-fat milk for 1 h. After washing with PBS-Tween, the membrane was incubated with the primary WNV antibody (15) at 1:3000 dilutions for 1 h at 25° C. Prior to incubation with HRP conjugated-mouset IgG secondary antibody (Amersham, Life Science, Piscataway, N.J.) diluted 1:5000, the blot was washed in PBS-Tween. Following the secondary antibody incubation, the blot was washed again and treated with Super Signal Chemiluminescent Reagent (Pierce) according to the manufacturer's protocol and exposed to X-ray film. For controls, the blots were stripped and re-probed with an antibody to beta-actin (Chemicon), which was detected with a goat anti-mouse horseradish peroxidase conjugate.

MTT Assays

Compound toxicity was determined by MTT assay. For the MTT assay, the cell medium was removed and the monolayers were incubated with 100 μl/well of 0.5 mg/ml MTT for 6 h at 37° C. The MTT solution was then aspirated and 100 μl/well of the 10% SDS (in 0.01N HCl) was added, followed by spectrophotometric quantitation (570 nm) of the insoluble MIT reaction product.

Northern Blot Analysis

Total cellular RNA was extracted by using the RNEASY kit (Qiagen NV, Netherlands). Northern blot analysis was done according to the protocol of Gu et al. 2002 (8). Briefly, 5 μg total RNA was electrophoresed through a 1.0% agarose gel containing 2.2M formaldehyde, transferred to a nylon membrane and immobilized by UV cross-linking (Stratagene, La Jolla, Calif.). After pre-hybridization in 5 ml of QuickHyb (Amersham, Piscataway, N.J. GE Healthcare, Giles, UK), [2P] dCTP-labeled probe made by random primer labeling of a 2.7 kb NS5 DNA fragment and a human beta-actin DNA at 65° C. The membrane was washed twice in 2×SSC/0.1% SDS for 10 min at room temperature and twice in 0.1×SSC/0.1% SDS for 15 min at 68° C. Membranes were exposed to Molecular Imager FX phosphoimager (BioRad, Hercules, Calif.) and the radiographic signals were collected and quantitated.

WNV Yield Reduction Assay

To measure activity against live WNV, BHK cells were plated in 96-well plates at a concentration of 12,000 cells/well. One day later the cells were infected with WNV for 1 h at an MOI of 0.05. The cells were then washed once and re-fed with fresh DMEM containing dilutions of the test compound. Plates were then incubated at 37° C. for 48 h, the supernatant collected and the WNV produced titered. For virus titration, Vero cells were plated in 96-well plates at 8000 cells/well and incubated overnight. The Vero cell monolayer were then infected for 1 h with various dilutions of the WNV supernatant, overlaid with media containing 0.6% tragacanth (ICN, CA) and incubated at 37° C. for 48 h. The culture media was then aspirated; the plate was rinsed, air-dried, and fixed with 50 μl/well acetone/methanol (50:50). Viral foci were detected for enumeration by immunostaining as described previously (15).

TABLE 3

| | | Fold decrease | Fold decrease | IC50 | Toxicity |
|---|---|---|---|---|---|
| Compound | Chemical name | (HTS) | (Re-test) | (uM) | (uM)* |
| 18-B3 | Thiophene-2-carboxylic acid | 3 | 2 | 5 | >30 |
| | [2-(4-methoxy-phenyl)-2,6- | | | | |
| | dihydro-4H-thieno[3,4- | | | | |
| | c]pyrazol-3-yl]-amide | | | | |
| 18-D2 | N-[2-(4-Methoxy-phenyl)- | 3 | 2.8 | 7.5 | >30 |
| | 2,6-dihydro-4H-thieno[3,4- | | | | |
| | c]pyrazol-3-yl]-4-(piperidine- | | | | |
| | 1-sulfonyl)-benzamide | | | | |
| 18-H5 | 4-(Piperidine-1-sulfonyl)-N- | 2 | 2 | 7.5 | >30 |
| | (2-m-tolyl-2,6-dihydro-4H- | | | | |
| | thieno[3,4-c]pyrazol-3-yl)- | | | | |
| | benzamide | | | | |
| 20-E7 | Cyclopropanecarboxylic acid | 2.3 | 2.4 | 10 | >10 |
| | [2-(2,4-dimethyl-phenyl)-2,6- | | | | |
| | dihydro-4H-thieno[3,4- | | | | |
| | c]pyrazol-3-yl]-amide | | | | |
| 253-B10 | N-[2-(3-Chloro-phenyl)-2,6- | 2 | 2 | 3 | >30 |
| | dihydro-4H-thieno[3,4- | | | | |
| | c]pyrazol-3-yl]-3,5- | | | | |
| | dimethoxy-benzamide | | | | |
| 253-F8 | 4-Isopropoxy-N-[2-(4- | 2.5 | 2 | 10 | >30 |
| | methoxy-phenyl)-2,6- | | | | |
| | dihydro-4H-thieno[3,4- | | | | |
| | c]pyrazol-3-yl]-benzamide | | | | |
| 253-F11 | Benzo[1,3]dioxole-5- | 3 | 2 | 10 | >30 |
| | carboxylic acid [2-(4-fluoro- | | | | |
| | phenyl)-2,6-dihydro-4H- | | | | |
| | thieno[3,4-c]pyrazol-3-yl]- | | | | |
| | amide | | | | |
| 253-G8 | Benzo[1,3]dioxole-5- | 2 | 2 | 2.5 | >30 |
| | carboxylic acid [2-(4- | | | | |
| | methoxy-phenyl)-2,6- | | | | |
| | dihydro-4H-thieno[3,4- | | | | |
| | c]pyrazol-3-yl]-amide | | | | |
| 253-H8 | 3,4-Diethoxy-N-[2-(4- | 2 | 1.5 | 10 | >30 |
| | methoxy-phenyl)-2,6- | | | | |
| | dihydro-4H-thieno[3,4- | | | | |
| | c]pyrazol-3-yl]-benzamide | | | | |
| 2-H7 | 3-(4-Chloro- | 4.5 | 2.5 | 2.5 | >20 |
| | benzenesulfonyl)- | | | | |
| | propionamide | | | | |
| 24-C10 | 2-Oxo-1,2-dihydro- | 2.5 | 1.8 | 10 | >10 |
| | benzo[cd]indole-6-sulfonic | | | | |
| | acid (2,5-dichloro-phenyl)- | | | | |
| | amide | | | | |
| 42-E5 | 2-(Toluene-4-sulfonyl)- | 7 | 1.7 | 7.5 | >10 |
| | 1,2,3,4-tetrahydro- | | | | |
| | isoquinoline-3-carboxylic | | | | |
| | acid(4-chloro-phenyl)-amide | | | | |
| 50-A8 | 6-Phenyl-2-(4,6,8-trimethyl- | 4.5 | 2.3 | 5 | >10 |
| | quinazolin-2-ylamino)-5,6- | | | | |
| | dihydro-3H-pyrimidin-4-one | | | | |
| 63-C10 | 4-Benzylamino-6-ethoxy- | 3.5 | ND | 5 | >20 |
| | quinoline-3-carboxylic acid | | | | |
| | ethyl ester | | | | |
| 101-G7 | N-(5-Methyl-isoxazol-3-yl)- | 6 | 2 | 5 | >10 |
| | 4-(4-p-tolyl-thiazol-2- | | | | |
| | ylamino)- | | | | |
| | benzenesulfonamide | | | | |
| 182-C2 | 1-(4-Cyclohexyl- | 5 | 1.4 | ND | >10 |
| | benzenesulfonyl)-3,5- | | | | |
| | dimethyl-piperidine | | | | |
| 207-E5 | N-(3-Ethyl-phenyl)-3-phenyl- | 5 | 2 | 10 | >10 |
| | 2-(thiophene-2- | | | | |
| | sulfonylamino)-propionamide | | | | |
| 275-D9 | 6-(2-Chloro-benzyl)-7- | 10 | 2.9 | 7.5 | >10 |
| | hydroxy-5-methyl- | | | | |
| | pyrazolo[1,5-a]pyrimidine-3- | | | | |
| | carboxylic acid sec- | | | | |
| | butylamide | | | | |
| 275-F9 | 6-(2-Chloro-benzyl)-7- | 27 | 4.5 | 7.5 | >10 |
| | hydroxy-5-methyl- | | | | |
| | pyrazolo[1,5-a]pyrimidine-3- | | | | |
| | carboxylic acid (2-cyclohex- | | | | |
| | 1-enyl-ethyl)-amide | | | | |
| 309-F6 | 6-(2-Fluoro-benzyl)-7- | 7 | 6 | 10 | >10 |
| | hydroxy-5-methyl- | | | | |
| | pyrazolo[1,5-a]pyrimidine-3- | | | | |
| | carboxylic acid [2-(ethyl- | | | | |
| | phenyl-amino)-ethyl]-amide | | | | |
| 310-B3 | 6-(2-Fluoro-benzyl)-7- | 17 | 4 | 10 | >10 |
| | hydroxy-5-methyl- | | | | |
| | pyrazolo[1,5-a]pyrimidine-3- | | | | |
| | carboxylic acid (2-p-tolyl- | | | | |
| | ethyl)-amide | | | | |
| 331-D11 | N-[4-(N- | 4 | 2 | 5 | >30 |
| | Hydroxycarbamimidoyl)- | | | | |
| | phenyl]-2-(4-methoxy- | | | | |
| | phenyl)-acetamide | | | | |
| 331-E11 | N-[4-(N- | 3 | 1.5 | 5 | >30 |
| | Hydroxycarbamimidoyl)- | | | | |
| | phenyl]-3-(4-methoxy- | | | | |
| | phenyl)-propionamide | | | | |

Methods of Making Compounds of the Invention

The compounds of Table 3 are available from the collections of Asinex Inc. (Moscow, Russia), Chembridge Inc. (San Diego, Calif.) and Maybridge Inc. (Cornwall, UK).

The flavivirus replication inhibitors of the invention and derivatives thereof can be prepared by methods and techniques known in the art. As can be appreciated by those skilled in the art, modifications and different substitutions of various groups in the compounds of the invention can be performed by known methods and techniques as for example, described in Strategic Applications of Named Reactions in Organic Synthesis: Background and Detailed Mechanisms by Laszlo Kurti ((2005); Academic Press; ISBN: 0124297854), U.S. Application Publication 2006/0040958 to Guzi et al., WO92/18504, WO02/50079, WO95/35298, WO02/40485, EP94304104.6, EP0628559 (equivalent to U.S. Pat. Nos. 5,602,136, 5,602,137 and 5,571,813), U.S. Pat. No. 6,383,790, Chem. Pharm. Bull., (1999) 47 928, J. Med. Chem., (1977) 20, 296, J. Med. Chem., (1976) 19 517 and Chem. Pharm. Bull., (1962) 10 620, WO01/92282 to Sommadossi et al., U.S. Pat. No. 6,812,219 to LaColla, which are incorporated herein in their entireties.

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, —NH(alkyl), —NH(cycloalkyl), —N(alkyl)₂, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine.

“Ring system substituent” means a substituent attached to an aromatic or non-aromatic ring system which, for example, replaces an available hydrogen group on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, and others as described in U.S. Application Publication 2006/0040958 to Guzi et al.

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like.

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridine, 1,2-dihydropyridyl, 1,4-dihydropyridyl, 1,2,3,6-tetrahydropyridine, 1,4,5,6-tetrahydropyrimidine, 2-pyrrolinyl, 2-imidazolinyl, 2-pyrazolinyl, dihydroimidazole, dihydrooxazole, dihydrooxadiazole, dihydrothiazole, 3,4-dihydro-2H-pyran, dihydrofuranyl, fluorodihydrofuranyl, 7-oxabicyclo[2.2.1]heptenyl, dihydrothiophenyl, dihydrothiopyranyl, and the like.

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring: there is no —OH attached directly to carbons marked 2 and 5.

It should also be noted that tautomeric forms such as, for example, the moieties: are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound of Formulas (I)-(XXIII) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by U.S. Application Publication 2006/0040958 to Guzi et al. referencing T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formulas (I)-(XXIII) can form salts which are also within the scope of this invention. Reference to a compound of Formulas (I)-(XXIII) herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formulas (I)-(XXIII) contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of Formulas (I)-(XXIII) may be formed, for example, by reacting a compound of Formulas (I)-(XXIII) with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quaternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C.sub.1-4alkyl, or C.sub.1-4alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a Cl₂₀ alcohol or reactive derivative thereof, or by a 2,3-di (C₆₋₂₄)acyl glycerol.

Compounds of F Formulas (I)-(XXIII) and salts, solvates, esters and prodrugs thereof, may exist in their tautomeric form (for example, as an amide or imino ether). All such tautomeric forms are contemplated herein as part of the present invention.

The compounds of Formulas (I)-(XXIII) may contain asymmetric or chiral centers, and, therefore, exist in different stereoisomeric forms. It is intended that all stereoisomeric forms of the compounds of Formulas (I)-(XXIII) as well as mixtures thereof, including racemic mixtures, form part of the present invention. In addition, the present invention embraces all geometric and positional isomers. For example, if a compound of Formulas (I)-(XXIII) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as achiral alcohol or Mosher's acid chloride), separating the diastereomers and converting (e.g., hydrolyzing) the individual diastereomers to the corresponding pure enantiomers. Also, some of the compounds of Formula (I), Formula (II), and Formula (III) may be atropisomers (e.g., substituted biaryls) and are considered as part of this invention. Enantiomers can also be separated, e.g., by use of chiral HPLC column.

It is also possible that the compounds of Formulas (I)-(XXIII) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I), Formula (II), and Formula (III) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

To ensure that the desired modified compounds have inhibiting abilities, these compounds will be tested as described herein. A variation in the inhibiting abilities is also contemplated.

Various modified compounds based on the compounds of Formulas (I)-(XXIII), will then be further tested to confirm the anti-WNV activity. Desired compounds would have anti-WNV activity with 50% inhibitory concentration (IC50) less than 5 μM and 50% cytotoxic concentration (CC 50) at greater than 50 μM in accordance with the live WNV virus assay as described below.

The compounds of the invention can be used as the starting point for further modification to make more potent compounds through structure activity relationship (SAR) studies. The structure of selected compounds can be modified through chemical synthesis. The compound derivatives will then be tested in WNV replicon cells. Changes than can increase the potency of the compound will be determined as important and can be further refined. Through such process more active compounds can be obtained. When a reasonably potent compound, for example, IC50 below 1 μM or low enough to be able to administer to animals is obtained, it can be evaluated in animal models for their activity against West Nile virus and other flavivirus infections including Dengue, yellow fever virus. These compounds can also be tested against other members of the flaviviridae family, e.g., Hepatitis C virus. Potentially, these compounds can be developed into small molecule antiviral therapeutics against many viruses in the flaviviridae family and possibly other viruses.

The compound can be further tested in a mouse model for WNV infection. There are available animal models that have been proven for this purpose (18).

While the invention has been described in detail and with reference to specific examples thereof, it will be apparent to one skilled in the art that various changes and modifications can be made therein without departing from the spirit and scope thereof.

### REFERENCES

1. Anderson, J. F., T. G. Andreadis, C. R. Vossbrinck, S. Tirrell, E. M. Wakem, R. A. French, A. E. Garmendia, and H. J. Van Kruiningen. 1999. Isolation of West Nile virus from mosquitoes, crows, and a Cooper's hawk in Connecticut. Science 286:2331-3.
2. Briese, T., X. Y. ha, C. Huang, L. J. Grady, and W. I. Lipkin. 1999. Identification of a Kunjin/West Nile-like flavivirus in brains of patients with New York encephalitis. Lancet 354:1261-2.
3. Brinton, M. A. 2001. Host factors involved in West Nile virus replication. Ann N Y Acad Sci 951:207-19.
4. Brinton, M. A. 2002. The molecular biology of West Nile Virus: a new invader of the western hemisphere. Annu Rev Microbiol 56:371-402.
5. Chambers, T. J., C. S. Hahn, R. Caller, and C. M. Rice. 1990. Flavivirus genome organization, expression, and replication. Annu Rev Microbiol 44:649-88.
6. Fauci, A. S., N. A. Touchette, and G. K. Folkers. 2005. Emerging infectious diseases: a 10-year perspective from the National Institute of Allergy and Infectious Diseases. Emerg Infect Dis 11:519-25.
7. Granwehr, B. P., K. M. Lillibridge, S. Higgs, P. W. Mason, J. F. Aronson, G. A. Campbell, and A. D. Barrett. 2004. West Nile virus: where are we now? Lancet Infect Dis 4:547-56.
8. Gu, B., A. T. Gates, O. Isken, S. E. Behrens, and R. T. Sarisky. 2003. Replication studies using genotype la subgenomic hepatitis C virus replicons. J Virol 77:5352-9.
9. Khromykh, A. A., P. L. Sedlak, K. J. Guyatt, R. A. Hall, and E. G. Westaway. 1999. Efficient trans-complementation of the flavivirus kunjin NS5 protein but not of the NS1 protein requires its coexpression with other components of the viral replicase. Virol 73:10272-80.
10. Lanciotti, R. S., J. T. Roehrig, V. Deubel, J. Smith, M. Parker, K. Steele, B. Crise, K. E. Volpe, M. B. Crabtree, J. H. Scherret, R. A. Hall, J. S. MacKenzie, C. B. Cropp, B. Panigrahy, E. Ostlund, B. Schmitt, M. Malkinson, C. Banet, J. Weissman, N. Komar, H. M. Savage, W. Stone, T. McNamara, and D. J. Gubler. 1999. Origin of the West Nile virus responsible for an outbreak of encephalitis in the northeastern United States. Science 286:2333-7.
11. Lo, M. K., M. Tilgner, and P. Y. Shi. 2003. Potential high-throughput assay for screening inhibitors of West Nile virus replication. J Virol 77:12901-6.
12. Monath, T. P., J. Arroyo, C. Miller, and F. Guirakhoo. 2001. West Nile virus vaccine. Curr Drug Targets Infect Disord 1:37-50.
13. Money, J. D., D. F. Smee, R. W. Sidwell, and C. Tseng. 2002. Identification of active antiviral compounds against a New York isolate of West Nile virus. Antiviral Res 55:107-16.
14. Pietschmann, T., V. Lohmann, G. Rutter, K. Kurpanek, and R. Bartenschlager. 2001. Characterization of Cell Lines Carrying Self-Replicating Hepatitis C Virus RNAs. J. Virol. 75:1252-1264.
15. Rossi, S. L., Q. Zhao, V. K. ODonnell, and P. W. Mason. 2005. Adaptation of West Nile virus replicons to cells in culture and use of replicon-bearing cells to probe antiviral action. Virology 331:457-70.
16. Shi, P. Y. 2002. Strategies for the identification of inhibitors of West Nile virus and other flaviviruses. Curr Opin Investig Drugs 3:1567-73.
17. Shi, P. Y., M. Tilgner, and M. K. Lo. 2002. Construction and characterization of subgenomic replicons of New York strain of West Nile virus. Virology 296:219-33.
18 Beasley, D. W., Li, L., Suderman, M. T., and Barrett, A. D. (2002) mouse neuroinvasive phenotype of West Nile virus strains varies depending upon virus genotype. Virology 296: 17-23.
19. Gu B, Ouzunov S, Wang L, Mason P, Bourne N, Cuconati A, Block TM. (2006). Discovery of small molecule inhibitors of West Nile virus using a high-throughput sub-genomic replicon screen. Antiviral Res. 70(2):39-50

What is claimed is:

1. A flavivirus replication inhibitor, wherein the flavivirus replication inhibitor is a compound of Formula (I) 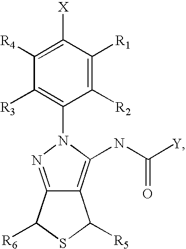
wherein X, Y and R₁₋₆ are members selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH.

2. The flavivirus replication inhibitor of claim 1, wherein the flavivirus replication inhibitor is an inhibitor of West Nile virus replication.

3. The flavivirus replication inhibitor of claim 2, wherein the flavivirus replication inhibitor has an anti-viral activity tested in a live virus assay with 50% inhibitory concentration (IC50) less than 5 μM and 50% cytotoxic concentration (CC 50) at greater than 50 μM.

4. The flavivirus replication inhibitor of claim 2, wherein the flavivirus replication inhibitor is a compound of Formula (XIII) 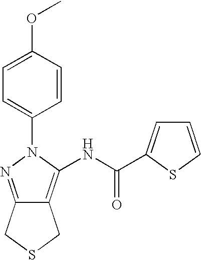
and derivatives thereof and pharmaceutically acceptable salts, solvates, esters or prodrugs thereof.

5. The flavivirus replication inhibitor of claim 2, wherein the flavivirus replication inhibitor is a compound of Formula (XIV) 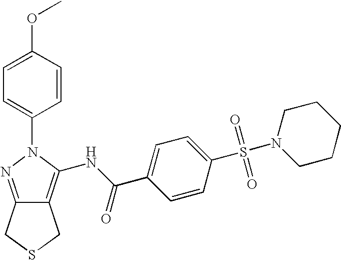
and derivatives thereof and pharmaceutically acceptable salts, solvates, esters or prodrugs thereof.

6. The flavivirus replication inhibitor of claim 2, wherein the flavivirus replication inhibitor is a compound of Formula (XV) 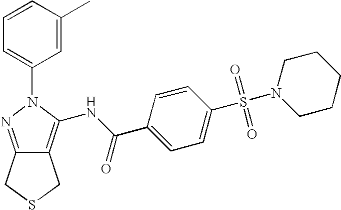
and derivatives thereof and pharmaceutically acceptable salts, solvates, esters or prodrugs thereof.

7. The flavivirus replication inhibitor of claim 2, wherein the flavivirus replication inhibitor is a compound of Formula (XVI) 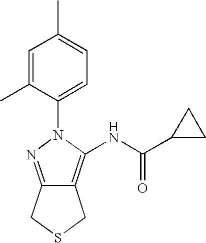
and derivatives thereof and pharmaceutically acceptable salts, solvates, esters or prodrugs thereof.

8. The flavivirus replication inhibitor of claim 2, wherein the flavivirus replication inhibitor is a compound of Formula (XVII) 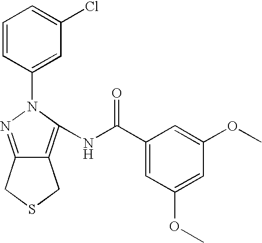
and derivatives thereof and pharmaceutically acceptable salts, solvates, esters or prodrugs thereof.

9. The flavivirus replication inhibitor of claim 2, wherein the flavivirus replication inhibitor is a compound of Formula (XVIII) 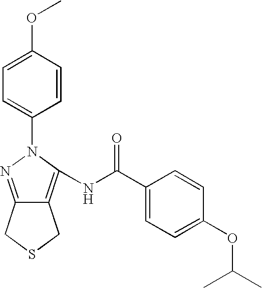
and derivatives thereof and pharmaceutically acceptable salts, solvates, esters or prodrugs thereof.

10. The flavivirus replication inhibitor of claim 2, wherein the flavivirus replication inhibitor is a compound of Formula (XIX) 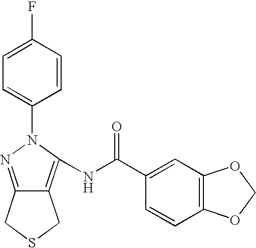
and derivatives thereof and pharmaceutically acceptable salts, solvates, esters or prodrugs thereof.

11. The flavivirus replication inhibitor of claim 2, wherein the flavivirus replication inhibitor is a compound of Formula (XX) 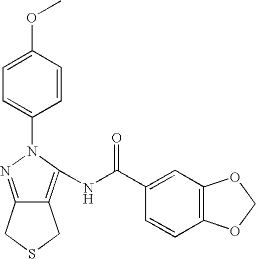
and derivatives thereof and pharmaceutically acceptable salts, solvates, esters or prodrugs thereof.

12. The flavivirus replication inhibitor of claim 2, wherein the flavivirus replication inhibitor is a compound of Formula (XXI) 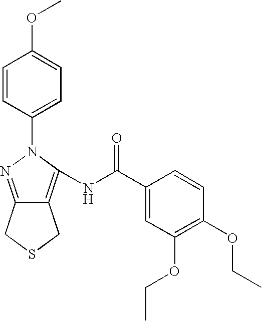
and derivatives thereof and pharmaceutically acceptable salts, solvates, esters or prodrugs thereof.

13. A pharmaceutical composition comprising a pharmaceutically acceptable carrier and a compound of claim 1.

14. A process for making a pharmaceutical composition comprising combining the flavivirus replication inhibitor of claim 1 and a pharmaceutically acceptable carrier.

15. A method of treatment, prevention, inhibition or amelioration of one or more diseases associated with flavivirus replication in a subject in need thereof comprising administering to the subject a therapeutically effective amount of a pharmaceutical composition comprising the flavivirus replication inhibitor of claim 1 and a pharmaceutically acceptable carrier.

16. The method of claim 15, wherein the flavivirus caused disorder is a disorder related to a West Nile virus caused disorder.

17. A flavivirus replication inhibitor, wherein the flavivirus replication inhibitor is a compound of Formula (II) 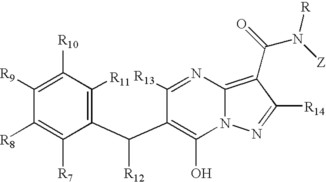
wherein R is a member selected from the group consisting of H, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH;
R₇ is a member selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH; and
R₈₋₁₄ are members selected from the group consisting of H, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH.

18. The flavivirus replication inhibitor of claim 17, wherein the flavivirus replication inhibitor is an inhibitor of West Nile virus replication.

19. The flavivirus replication inhibitor of claim 18, wherein the flavivirus replication inhibitor has an anti-viral activity tested in a live virus assay with 50% inhibitory concentration (IC50) less than 5 μM and 50% cytotoxic concentration (CC 50) at greater than 50 μM.

20. The flavivirus replication inhibitor of claim 18, wherein the flavivirus replication inhibitor is a compound, of Formula (XI) 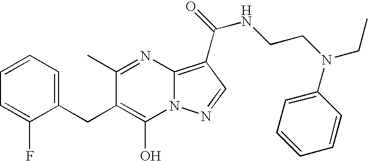
and derivatives thereof and pharmaceutically acceptable salts, solvates, esters or prodrugs thereof.

21. The flavivirus replication inhibitor of claim 18, wherein the flavivirus replication inhibitor is a compound of Formula (XII) 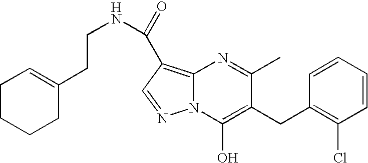
and derivatives thereof and pharmaceutically acceptable salts, solvates, esters or prodrugs thereof.

22. The flavivirus replication inhibitor of claim 18, wherein the flavivirus replication inhibitor is a compound of Formula (XXII) 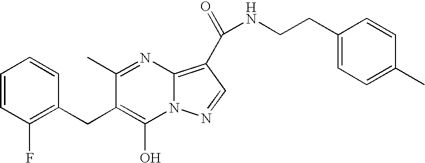
and derivatives thereof and pharmaceutically acceptable salts, solvates, esters or prodrugs thereof.

23. A pharmaceutical composition comprising a pharmaceutically acceptable carrier and the flavivirus replication inhibitor of claim 17.

24. The pharmaceutical composition of claim 23, wherein the flavivirus replication inhibitor is the compound of Formula (XI) 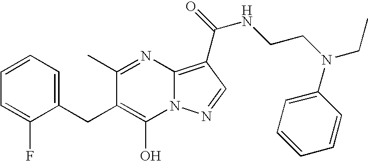
and derivatives thereof and pharmaceutically acceptable salts, solvates, esters or prodrugs thereof.

25. A process for making a pharmaceutical composition comprising combining the flavivirus replication inhibitor of claim 17 and a pharmaceutically acceptable carrier.

26. A method of treatment, prevention, inhibition or amelioration of one or more diseases associated with flavivirus replication in a subject in need thereof comprising administering to the subject a therapeutically effective amount of a pharmaceutical composition comprising the flavivirus replication inhibitor of claim 17 and a pharmaceutically acceptable carrier.

27. The method of claim 26, wherein the flavivirus caused disorder is a disorder related to a West Nile virus caused disorder.

28. A flavivirus replication inhibitor, wherein the flavivirus replication inhibitor is a compound of Formula (III) 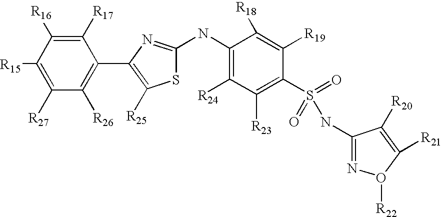
wherein R₁₅₋₂₇ are members selected from the group consisting of H, halogen, alkyl, aryl, alkoxy, arylalkyl, cycloalkyl, heterocyclyl, heterocyclylalkyl, heteroaryl, and heteroarylalkyl, wherein each of said alkyl, aryl, alkoxy, arylalkyl, cycloalkyl, heterocyclyl, heterocyclylalkyl, heteroaryl, and heteroarylalkyl can be unsubstituted or optionally substituted with one or more moieties which can be the same or different, each moiety being independently selected from the group consisting of halogen, alkyl, aryl, arylalkyl, cycloalkyl, alkoxy, heterocyclyl, heterocyclylalkyl, heteroaryl, heteroarylalkyl, CF₃, OCF₃, CN, and —OH.

29. The flavivirus replication inhibitor of claim 28, wherein the flavivirus replication inhibitor is an inhibitor of West Nile virus replication.

30. The flavivirus replication inhibitor of claim 29, wherein the flavivirus replication inhibitor has an anti-viral activity tested in a live virus assay with 50% inhibitory concentration (IC50) less than 5 μM and 50% cytotoxic concentration (CC 50) at greater than 50 μM.

31. The flavivirus replication inhibitor of claim 29, wherein the flavivirus replication inhibitor is a compound of Formula (IV) 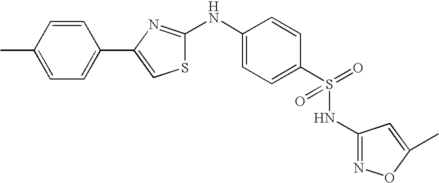
and derivatives thereof and pharmaceutically acceptable salts, solvates, esters or prodrugs thereof.

32. A pharmaceutical composition comprising a pharmaceutically acceptable carrier and the flavivirus replication inhibitor of claim 28.

33. The pharmaceutical composition of claim 32, wherein the flavivirus replication inhibitor is the compound of Formula (IV) 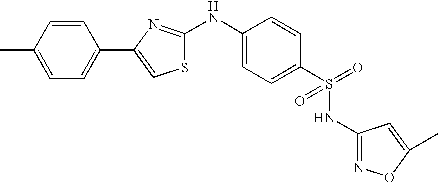
and derivatives thereof and pharmaceutically acceptable salts, solvates, esters or prodrugs thereof.

34. A process for making a pharmaceutical composition comprising combining the flavivirus replication inhibitor of claim 28 and a pharmaceutically acceptable carrier.

35. A method of treatment, prevention, inhibition or amelioration of one or more diseases associated with flavivirus replication in a subject in need thereof comprising administering to the subject a therapeutically effective amount of a pharmaceutical composition comprising the flavivirus replication inhibitor of claim 28 and a pharmaceutically acceptable carrier.

36. The method of claim 35, wherein the flavivirus caused disorder is a disorder related to a West Nile virus caused disorder.

37. A flavivirus replication inhibitor, wherein the flavivirus replication inhibitor is at least one of
(a) a compound of Formula (V) 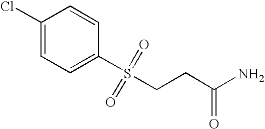
and derivatives thereof and pharmaceutically acceptable salts, solvates, esters or prodrugs thereof;
(b) a compound of Formula (VI) 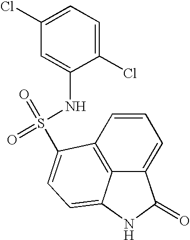
and derivatives thereof and pharmaceutically acceptable salts, solvates, esters or prodrugs thereof;
(c) a compound of Formula (VII) 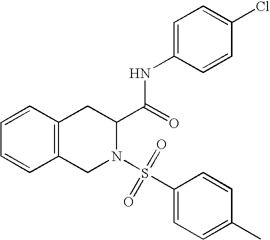
and derivatives thereof and pharmaceutically acceptable salts, solvates, esters or prodrugs thereof;
(d) a compound of Formula (VIII) 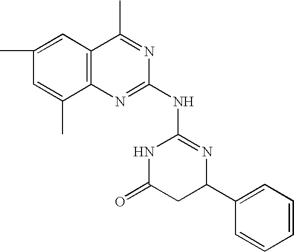
and derivatives thereof and pharmaceutically acceptable salts, solvates, esters or prodrugs thereof;
(e) a compound of Formula (IX) 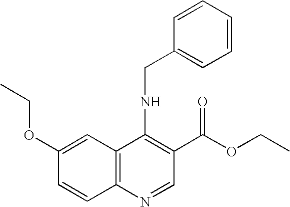
and derivatives thereof and pharmaceutically acceptable salts, solvates, esters or prodrugs thereof;
(f) a compound of Formula (X) 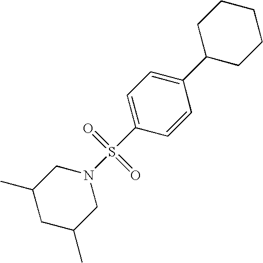
and derivatives thereof and pharmaceutically acceptable salts, solvates, esters or prodrugs thereof; or
(g) a compound of Formula (XXIII) 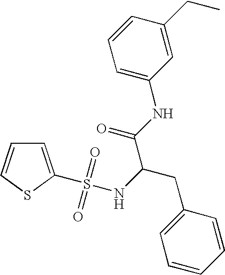
and derivatives thereof and pharmaceutically acceptable salts, solvates, esters or prodrugs thereof.
